(19) United States
(12) Patent Application Publication
(10) Pub. No.: US 2005/0048491 A1
(43) Pub. Date: Mar. 3, 2005

(54) Methods and kits for use in selecting approaches to treating cancer

(76) Inventors: Ralph R. Weichselbaum, Chicago, IL (US); Bernard Roizman, Chicago, IL (US); Nikolai N. Khodarev, Villa Park, IL (US); Nalin Gupta, San Francisco, CA (US)

Correspondence Address: CLARK & ELBING LLP, 101 FEDERAL STREET, BOSTON, MA, 02110, US

(21) Appl. No.: 10/492,239

(22) PCT Filed: Oct. 9, 2002
(86) PCT No.: PCT/US02/32146

Related U.S. Application Data
(60) Provisional application No. 60/328,078, filed on Oct. 9, 2001.

(51) Int. Cl. C12Q 1/68; G01N 33/53; G01N 33/574

(52) U.S. Cl. 435/6; 435/7.23

(57) ABSTRACT

This invention provides methods and kits for use in selecting approaches to treating cancer, as well as methods for identifying genes that can be used in such methods and kits.

### FIELD OF THE INVENTION

This invention relates to methods and kits for use in selecting approaches to treating cancer.

### BACKGROUND OF THE INVENTION

Ionizing radiation (IR) has been used for nearly a century to treat human cancer (Hall, Radiobiology for Radiologists, 5^{th} edition, Lippincott, Williams, and Wilkins, Philadelphia, p. 5-17, 2000). The objective of IR therapy is to deliver a lethal dose of IR to cancer cells, while at the same time minimizing the toxic effects of IR on adjacent normal tissue. Undesirable consequences of radiotherapy include the development of tumor resistance and normal tissue damage (Viayakumar et al., Lancet 349:1-30, 1997).

Various types of DNA damage, including IR, are recognized and repaired by specialized pathways that were first described in prokaryotes. Many of the genes involved in DNA repair are conserved (Takanami et al., Nucleic Acids Res. 28:4232-4236, 2000; Saintigny et al., EMBO J. 20:3861-3870, 2001). Transcriptional induction of DNA repair genes, immediate early genes, and a variety of cytokine and growth factor genes has been proposed as a mechanism that facilitates survival of cells following IR (Hallahan et al., Proc. Natl. Acad. Sci. U.S.A. 86:10104-10107, 1989; Witte et al., Cancer Res. 49:5066-5072, 1989). Gene induction following the exposure of mammalian cells to IR has been reported (Komarova et al., Oncogene 17:1089-1096, 1998; Zhao et al., Genes Dev. 14:981-993, 2000; Amundson et al., Oncogene 18:3666-3672, 1999; Tusher et al., Proc. Natl. Acad. Sci. U.S.A. 98:5116-5121, 2001). These reports describe induction of genes by IR in general terms, without reference to doses employed in radiotherapy or the timing of gene induction. Moreover, many in vitro studies of gene induction were performed under supra-physiologic IR doses, and therefore would be of limited value in the design of potential treatments.

### SUMMARY OF THE INVENTION

In a first aspect, the invention provides methods of selecting approaches to treating cancer in subjects using radiation therapy. These methods involve (i) analyzing the level of expression of one or more cancer-associated genes in a sample containing cancer cells from a subject, and (ii) selecting a type, schedule, route, and/or amount of radiation therapy for treating the subject based on the results of the analysis.

The subject may or may not have previously been treated using radiation therapy, or may have previously received cancer treatment not involving radiation therapy. Also, the methods can be used to indicate the use of a treatment in addition to radiation therapy; predict the outcome of treatment, such as treatment involving radiation therapy; or allow modification of radiotherapy during treatment.

The methods of the invention can involve detection of an increase in expression of a gene associated with resistance to radiation therapy, or a decrease in expression of a gene associated with sensitivity to radiation therapy. In such cases, it can be determined that a radiosensitizer should be administered to a subject. The time frame of such administration can also be determined, based on analysis of the temporal expression of the gene associated with resistance to radiation therapy or the gene associated with sensitivity to radiation therapy. Moreover, the dosage at which the radiosensitizer is to be administered to the subject can be determined by analysis of the level of expression of the gene associated with resistance to radiation therapy or the gene associated with sensitivity to radiation therapy. The methods can also involve detection of an increase in expression of one or more genes associated with sensitivity to radiation therapy, or a decrease in expression of one or more genes associated with resistance to radiation therapy, indicating treatment using further radiation therapy.

In a second aspect, the invention provides methods of selecting approaches to treating cancer in subjects that have previously been treated using radiation therapy. These methods involve (i) analyzing the level of expression of a cancer-associated gene in a sample containing cancer cells from a subject, and (ii) selecting a type, schedule, route, and/or amount of a therapy not involving further radiation therapy for treating the subject based on the results of the analysis. In any of the methods described above, the non-radiation therapy can be, for example, selected from the group consisting of chemotherapy, biological therapy, gene therapy, oncolytic viral therapy, and surgery. Examples of types of chemotherapeutic agents that can be indicated include alkylating agents, antineoplastic antibiotics, antimetabolites, and natural source derivatives, and specific examples of each of these types of chemotherapeutic agents are as follows: alkylating agents: busulfan, caroplatin, carmustine, chlorambucil, cisplatin, cyclophosphamide, dacarbazine, ifosfamide, lomustine, mecholarethamine, melphalan, procarbazine, streptozocin, and thiotepa; antineoplastic antibiotics: bleomycin, dactinomycin, daunorubicin, doxorubicin, idarubicin, mitomycin, mitoxantrone, pentostatin, and plicamycin; antimetabolites: fluorodeoxyuridine, cladribine, cytarabine, floxuridine, fludarabine, flurouracil, gemcitabine, hydroxyurea, mercaptopurine, methotrexate, and thioguanine; and natural source derivatives: docetaxel, etoposide, irinotecan, paclitaxel, teniposide, topotecan, vinblastine, vincristine, vinorelbine, taxol, prednisone, and tamoxifen.

An example of a type of biological therapeutic agent that can be used is immunomodulatory molecules, such as cytokines, chemokines, complement components, complement component receptors, immune system accessory molecules, adhesion molecules, and adhesion molecule receptors. Specific examples of cytokines include, for example, interleukins, interferons, tumor necrosis factor, granulocyte macrophage colony stimulating factor, macrophage colony stimulating factor, and granulocyte colony stimulating factor.

In any of the methods described herein, the expression of more than one cancer-associated gene can be analyzed and, as is discussed in further detail below, this analysis can be carried out using a nucleic acid molecule array. Also, in any of the methods described herein, the analysis can involve determination of the level of expression of a gene at more than one time point after any prior treatment. Such an analysis can be used to indicate an optimal time frame during which a particular type of subsequent treatment should be carried out. Moreover, the method can involve analyzing the effects of varying doses of a prior treatment, to indicate an optimal dosage at which a particular type of subsequent treatment should be carried out. Further, the methods described herein can be carried out on a tumor sample ex vivo or on a tumor in vivo. Examples of cancers that can be analyzed and treated using the methods and kits of the invention include lung, prostate, ovarian, testicular, brain, skin, colon, gastric, esophageal, tracheal, head and neck, pancreatic, liver, breast, lymphoid, cervical, vulvar, mesothelial, connective tissue, and epithelial cell cancers. In addition; examples of cancer-associated genes that can be analyzed using the present methods and kits can be selected from the group consisting of those involved in cell adhesion, cell death, cell cycle, cell maintenance, cell metabolism, protein synthesis, degradation pathways, DNA synthesis, RNA synthesis, RNA metabolism, DNA repair, and apoptosis. Numerous specific examples of such genes are known in the art (also see the tables provided herein).

In a third aspect, the invention provides methods of treating cancer in a subject using an approach selected by using any of the methods described herein. These methods can be based on analysis of a tumor sample from the subject to be treated or can, rather, be based on previous analyses of similar tumor samples from other subjects. Thus, once the parameters for a specific class of tumors is established, it may not necessarily be required to analyze samples from every subject having that class of tumor. Rather, once a tumor has been identified as being of a particular class, e.g., by immunohistochemistry or other methods, an approach to treatment based on expression analysis of similar tumors from other subjects can be used.

In a fourth aspect, the invention provides kits for use in selecting approaches to treating cancer in subjects. The kits can include one or more cancer-associated gene probes, as well as instructions to hybridize the probes with nucleic acid molecules derived from a tumor sample from a subject, to determine the level of expression of the gene in the tumor as an indication of an appropriate type, schedule, and/or amount of therapy to use in the treatment. The kit can include more than one cancer-associated gene probe, and the probes can be immobilized on a solid support, e.g., in an array.

In a fifth aspect, the invention provides methods of identifying genes that can be used in the identification of an approach to treating cancer in subjects. These methods involve contacting a nucleic acid molecule array with cDNA or RNA derived from a sample from a tumor of a subject, and detecting altered levels of binding of the tumor sample-derived cDNA or RNA to a position in the array, relative to a control. The identity of the gene that corresponds to the position in the array can then be determined. The tumor can previously have received anticancer treatment. A sample from the tumor prior to treatment can be used as a control.

The invention provides several advantages. For example, the invention facilitates the selection of treatment protocols that are tailored for a particular patient, as well as the modification and fine-tuning of such protocols during the course of treatment, based on the patient's response. The approaches to treatment that are selected using the methods and kits of the invention can lead to increased safety, efficacy, and comfort in the treatment of a patient. For example, if a tumor is found not to be susceptible to a particular type of therapy (e.g., ionizing radiation) using the methods of the invention, use of that type of therapy can be ruled out and a more appropriate type of therapy selected. This type of analysis can be carried out before any type of treatment or after treatment has occurred. The invention also facilitates the selection of appropriate amounts, routes, and schedules of therapy to maximize efficacy, while minimizing the untoward side effects that can accompany certain types of cancer therapy. For example, detection of an increased amount expression of a gene that indicates susceptibility to a particular treatment during a particular time period indicates an optimal time for using that type of treatment. After such treatment, if the level of expression of the gene has decreased, then another choice can be made, based on the current level of expression of another gene or genes associated with either resistance or sensitivity to another treatment. The invention also provides methods for identifying additional genes that can be used as indicators of resistance or sensitivity to treatment, which thus provide the opportunity for additional fine-tuning of therapeutic methods.

Other features and advantages of the invention will be apparent from the following detailed description, the claims, and the drawings.

### BRIEF DESCRIPTION OF THE DRAWINGS

FIG. 1 is a series of scatterplots of intensity values of independent GeneFilters® GF211 arrays that were hybridized with different samples of RNA. DNA arrays were hybridized, and data were acquired, with ImageQuant® and normalized as is described in the Materials and Methods section, below. Intensity values of one array plotted versus intensity values of the same genes on another array are shown. Left panels: U87 in vitro, right panels: U87 in vivo. Upper panels: scatterplots of two arrays, hybridized with the same mock (un-irradiated) sample of RNA. Numbers at the left bottom comer of panels: cut off values for intensities (see Methods, below). Lower left panels: intensity values of RNA from U87 cell cultures exposed to 1, 3, and 10 Gy, extracted from cells harvested 5 hours after irradiation and plotted versus corresponding values of RNA from in vitro mock-irradiated cells (see upper left panel). Lower right panels: intensity values of RNA from U87 xenografts exposed to 10 Gy, and extracted from tissues harvested at 1, 5, and 24 hours after irradiation and plotted versus corresponding RNA from in vivo mock-irradiated cells (see upper right panel).

FIG. 2 is a series of graphs showing a representation of temporal patterns of gene expression following IR of U87 xenografts in mice. The xenografts were exposed to 1, 3, and 10 Gy and collected at 1, 5, or 24 hours after irradiation. U87 genes that responded both in irradiated xenografts and in culture (Table 3) were grouped in 6 clusters (Panels A-F). Panels AG-FG show the distribution of functional groups in each cluster. In clusters A-D the temporal response was dose independent, although the magnitude of the response was in part dose dependent. Green, blue, and red corresponds to 1, 3, and 10 Gy, respectively. The black line shows the mean value for the entire cluster. Mean values of induction for each cluster is presented by gray line and colored lines present examples of individual responses for glutamate-cysteine ligase (panel A), protein phosphatase 2A (panel B), and phospholemman chloride channel (panel C). In clusters E-F the temporal pattern of gene response was dose dependent. Colored lines correspond to mean values of entire clusters at each dose tested. The genes are identified in Table 3.

FIG. 3 is a graph showing that radiation-induced transcriptional changes of FAS receptor (Apo-1, CD-95) gene expression coincide with FAS ligand-induced cytotoxicity in HUVE cells that have been treated with irradiation (9 Gy) at the time points indicated.

### DETAILED DESCRIPTION

The invention is based, in part, on our observation that, within a range of cytoreductive doses of ionizing radiation (IR) administered in clinical practice, gene expression responses to IR are dose-dependent and vary over time following IR treatment. The invention thus provides methods and kits for use in determining rational approaches to radiotherapy and other methods of treating cancer, based on the analysis of gene expression profiles of cancer cells from patients before, during, or after IR treatment. The invention facilitates selection of particular types, schedules, routes, or amounts of appropriate therapies for treating subjects, such as human patients. The methods of the invention can also be used with animal subjects (e.g., livestock, non-human primates, or laboratory animals), either for actual treatment or for preclinical identification and characterization of treatment protocols. The invention also provides methods for identifying genes that are associated with resistance or sensitivity of tumors to treatment, such as, for example, radiation treatment. These genes and their products can then be used as targets in cancer treatment. Kits for carrying out the methods described herein are also included in the invention. The methods and kits of the invention are described further, as follows.

In general, the methods of the invention involve analysis of the expression of genes in cancer cells before, during, and/or after IR treatment. Based on the detection of certain levels of expression of particular genes in cancer cells, medical professionals can select appropriate approaches to treating the cancer. In addition, such analysis can be used to enable medical professionals to predict the outcome of therapy, such as IR therapy, prior to treatment. Further, this analysis can be used to assist in determining whether an ongoing course of treatment should be modified by, e.g., changing the amount or duration of treatment, or by adding or removing a type of treatment.

In one example of a method of the invention, detection of expression of one or more genes associated with resistance to a particular type of primary treatment (e.g., IR treatment) can indicate to a medical professional that an additional type of therapy, such as one that increases sensitivity to the primary treatment, should be carried out. The detection of expression of genes associated with resistance to a particular type of treatment, such as IR treatment, can also indicate the use of a different type of treatment altogether. As an example, if one type of treatment leads to the induction of expression of genes associated with cell growth (e.g., genes encoding proteins involved in DNA, RNA, or protein synthesis), another type of treatment, which counteracts the activities of these genes, thus leading to inhibition of cell growth and cancer cell death, can be indicated. Selection of this different type of treatment can also be facilitated by gene expression analysis. For example, at the same time that induction of expression of a gene (or genes) associated with resistance to a particular type of therapy is detected, thus possibly indicating cessation of that therapy, induction of expression of another gene or genes associated with susceptibility to another type of treatment can be detected, thus indicating use of the other type of therapy.

In another example of a method of the invention, the detection of induction or suppression of expression of genes associated with susceptibility to a particular type of treatment can be used as an indication that the therapy should be used or, if already in use, continued. As a specific example of this method of the invention, detection of the induction of expression of genes associated with cell death in tumor cells that have received a particular type of treatment (e.g., ionizing radiation) can indicate that the current course of treatment is effective and should be continued.

Central to the methods of the invention is the determination of optimal timing and/or dosing of a particular treatment. For example, if a gene that is associated with susceptibility to a particular treatment is determined to have a peak in expression at a certain time point after the same or a different type of treatment (e.g., IR treatment), then a medical professional can use this information to choose the optimal time during which to administer the treatment associated with expression of the gene. Thus, the treatment can be administered during the peak level of expression, to obtain maximal effect, while minimizing the exposure of the patient to the treatment during time periods when it would be less effective.

Further, the methods of the invention can be used to determine the optimal dosing of treatments. For example, as is shown in Tables 3 and 4, below, we have found that different genes are induced to different levels in response to different amounts of IR treatment. Thus, the methods of the invention can be used in the identification of particular treatment dosages that lead to the induction or suppression of expression of genes that are indicative of sensitivity to treatment with the same or another treatment. Thus, for example, a level of ionizing radiation treatment can be identified that results in expression of such genes, and treatment with a therapeutic approach that expression of these genes indicates can be carried out. Alternatively, for example, if induction of expression of a gene associated with resistance is detected in response to a particular level of treatment, then the level of the treatment can be decreased. The methods of the invention thus can be used to determine optimal daily doses, as well as overall necessary doses, to treat a particular patient. Similarly, the methods of the invention can be used to determine whether different modes of administration should be used.

The genes analyzed using the methods of the invention can be analyzed for their induction or suppression of expression, both of which can be indicative of appropriate approaches to therapy. The genes can be by their very nature indicative of a particular further treatment to be used. For example, as is mentioned above, genes that are associated with cell death and induced using a particular treatment can be used as indicators that the treatment should be continued. Similarly, suppression of genes that are involved in cell growth can be indicative that the treatment that led to the suppression should be continued. In another example, if it is desirable to suppress the expression of an induced gene or to suppress the activity of the product of the gene in the treatment of cancer, an appropriate treatment, such as a small molecule, antibody, or antisense molecule that results in such suppression, can be administered to a patient. Conversely, if it is desirable to increase the expression of a suppressed gene or to increase the activity of the product of such a gene, an appropriate treatment that results in the desired effect can be administered.

A gene that is induced or suppressed by a particular treatment may not itself have an effect on tumor growth, but its level of expression (e.g., in response to prior treatment) can be used in therapeutic approaches, nonetheless, as agents that target therapeutics to cells expressing these genes can be used in therapy. For example, if it is found that IR treatment leads to induction of expression of a particular gene at a particular time point after treatment in a tumor cell, treatment can involve administration of an antibody or other molecule specific for the product of the gene. Such an antibody, which is targeted to the tumor cell, can be linked to an agent that kills the tumor cell.

The methods of the invention can be carried out by contacting nucleic acid molecule arrays with material obtained or derived from patient samples, and detecting the levels of expression of particular genes in the samples by analysis of hybridization of the material to positions on the arrays that include probes that correspond to particular genes. Any of a number of commercially available nucleic acid molecule arrays can be used in the invention. For example, GeneFilters® GF211 cDNA arrays (Research Genetics) can be used, and details concerning the use of these arrays are provided below. Other examples of commercially available arrays that can be used in the invention are Affymetrix® GeneChip® arrays. Alternatively, those of skill in this art can synthesize their own arrays for use in the invention, using methods that are standard in the art (see, e.g., U.S. Pat. Nos. 6,218,122; 5,412,087; 4,681,870; 5,601,980; 4,542,102; 4,937,188; 5,011,770; 5,436,327; and 5,143,854; as well as Fodor et al., Science 251:767-773, 1991; and Dower et al., Ann. Rev. Med. Chem. 26:271-280, 1991). The key feature of these arrays, for use in the invention, is that nucleic acid molecule probes corresponding to certain genes are positioned in the arrays at known locations, so that detection of hybridization of cDNA or RNA derived from patient samples to the arrays in these locations can be used as quantifiable indications of expression of the genes corresponding to these probes. Methods for using these arrays in the quantification and analysis of gene expression patterns are well known in the art (see, e.g., U.S. Pat. No. 6,218,122).

Selection of appropriate approaches to treatment, based on the expression patterns of genes, such as those listed in Tables 3 and 4 (below) or genes identified using the methods described herein, can be carried out by those of skill in this art, using the methods of the invention. For example, as is shown in Table 4, we have shown that expression of the epidermal growth factor receptor (EGFR) gene is induced in response to IR treatment, and that expression of this gene increases over time after IR treatment. Expression of this gene has been observed to be associated with resistance to therapy, such as IR therapy. Thus, detection of the induction of expression of this gene can be used as an indication that an additional type of therapy, such as one that increases sensitivity to radiation, should be carried out. For example, in such circumstances, an antibody, such as a monoclonal antibody, that blocks the activity of the EGFR receptor (e.g., IMC-C225; Imclone) can be administered to increase sensitivity to IR therapy. Similarly, a small molecule that inhibits the tyrosine kinase domain of the EGFR can be administered to increase sensitivity to IR therapy. Further, an antisense molecule against EGFR can be used to increase sensitivity. The optimal timing of this therapy can also be determined using the methods of the invention. For example, because the levels of the EGFR steadily increased during the time points that we analyzed, 1, 5, and 24 hours, those of skill in this art could conclude that the sensitizing therapy should be administered, e.g., between 5 and 24 hours after IR treatment.

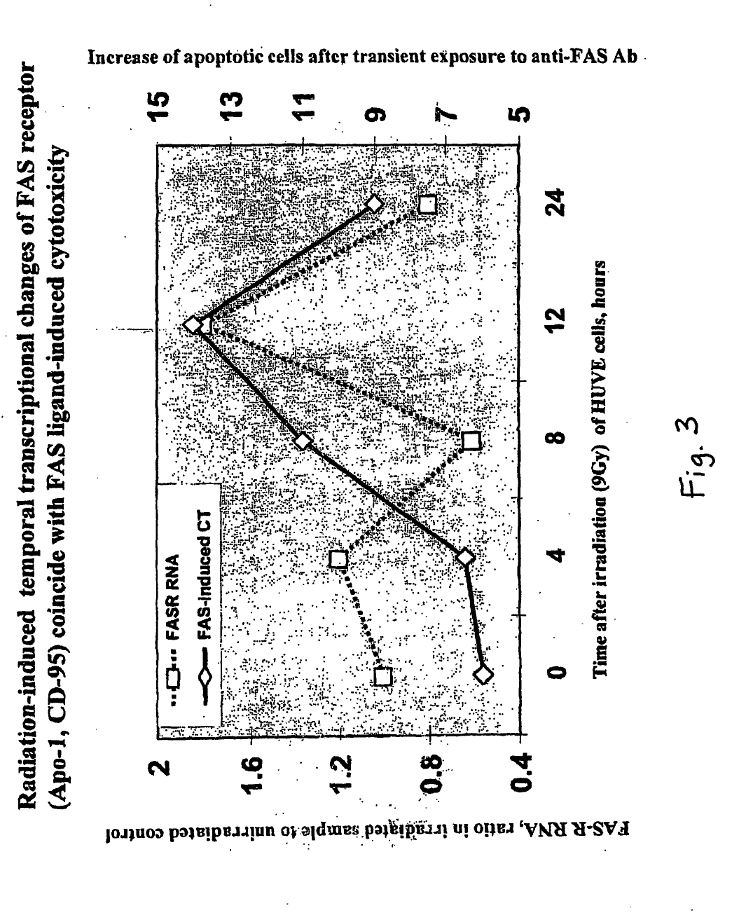

Another specific example of the methods of the invention is described further below and is illustrated in FIG. 3. Briefly, binding of the Fas ligand to the Fas receptor is known to induce apoptosis. We observed that expression of the Fas receptor is induced by IR treatment, peaking at about 12 hours after treatment. We also observed that induction of cytoxicity by the Fas ligand peaked at this time. Our observations show that, under the conditions of our study, the optimal time frame during which the Fas ligand can be administered to a patient after IR treatment to induce cancer cell death is around 12 hours after IR. These observations thus facilitate the rational design of treatment protocols, based on detection of expression of the Fas ligand.

As a further example, as is shown in Table 4, we found that the breast cancer 1, early onset gene is induced early after IR treatment, at 1, 3, and 10 Gy, and that expression of this gene decreases over time after the initial induction. These observations indicate that use of a therapeutic approach that affects the breast cancer 1, early onset gene could best be carried out early after IR treatment. The product of this gene, similar to p53, plays a role in mediating cell death. Thus, a therapeutic approach selected using the methods of the invention can focus on enhancing the activities of these proteins in tumors, while blocking their activities in normal tissues. Moreover, detection of abnormally low levels of these proteins can indicate the use of a radiosensitizer. Similarly, we found that expression of certain genes involved in DNA repair, such as rad51, increases over time after IR treatment (Table 4). Thus, a medical professional could determine that therapeutic approaches directed at inhibiting the activity of such genes would best be administered some time well after IR treatment (e.g., 24 hours later), as opposed to immediately after such treatment.

Patient samples for use in the methods of the invention can be obtained using standard methods, which will vary depending on the type of cancer that is being analyzed, and can readily be selected by those of skill in the art. As a specific example, needle aspiration can be used to obtain samples from many different types of tumors. In the case of a hematological malignancy, a sample can simply be obtained by blood withdrawal. Other tumor samples can be obtained during the course of a surgical procedure that is being conducted in an attempt to destroy or remove a tumor from a patient.

Material from patient samples can be prepared for use in the methods of the present invention using standard techniques. Preferably, mRNA is isolated from the samples and the isolated mRNA is then used as a template for the synthesis of cDNA (see, e.g., Chirgwin et al., Biochemistry 18:5294-5299, 1979; Sambrook et al., Molecular Cloning—A Laboratory Manual (2^{nd} Edn.), Vol. 1-3, Cold Spring Harbor Laboratory Press, Cold Spring Harbor, N.Y., 1989). The cDNA can be labeled, either during or after synthesis, and then contacted with an array for detection of gene expression within the sample. Any of a number of standard labels can be used such as, for example, fluorescent or radioactive labels, and methods for incorporating such labels into nucleic acid molecules are well known in the art (see, e.g., Klug et al., Methods Enzymol. 152:316-325, 1987).

Numerous methods, equipment, and software for use in detecting the presence of labeled nucleic acid molecules on particular regions of arrays, as well as for quantifying such labels, are well known in the art and can be used in the present invention. See, e.g., below, where use of the PATHWAYS 2.01® software package with GeneFilters® GF211 arrays is described. Also described below is the use of a Storm 860 Phosphorimager and IMAGEQUANT (Molecular Dynamics). Additional systems that can be used in the invention, such as laser scanners (e.g., the ScanArray 3000, General Scanning), are well known in the art. Also see, for example, the methods and equipment described in U.S. Pat. No. 6,218,122.

As is noted above, the methods of the invention can be carried out using samples from tumors that have not received any type of treatment, to get a baseline reading of gene expression and to use this baseline to select an appropriate mode of treatment. Optionally, after the use of a treatment, an additional reading or several readings can be taken to determine whether the selected mode of treatment should be continued or altered (e.g., the dosage and/or timing changed), or whether another approach to treatment should be used. The methods can also be carried out using samples from tumors that have received treatment, but have not yet received a reading of their gene expression, according to the invention. Further, the methods can be used to monitor expression during the course of treatment.

The methods of the invention can be used in conjunction with all types of cancers. Examples of cancers that can be analyzed for treatment using the methods of the invention include cancers of nervous system, for example, astrocytoma, oligodendroglioma, meningioma, neurofibroma, glioblastoma, ependymoma, Schwannoma, neurofibrosarcoma, neuroblastoma, pituitary tumors (e.g., pituitary adenoma), and medulloblastoma. Other types of cancers that can be analyzed using the methods of the invention include, head and neck cancer, melanoma, prostate carcinoma, renal cell carcinoma, pancreatic cancer, breast cancer, lung cancer, colon cancer, gastric cancer, bladder cancer, liver cancer, bone cancer, fibrosarcoma, squamous cell carcinoma, neurectodermal, thyroid tumor, lymphoma (Hodgkin's and non-Hodgkin's lymphomas), hepatoma, mesothelioma, epidermoid carcinoma, cancers of the blood (e.g., leukemias), as well as other cancers mentioned herein.

The methods of the invention can be used in the selection of any type of cancer therapy, as is understood in the art. Of course, any treatment selected using the methods of the invention is preferably as specific for cancer cells as possible, minimizing adverse effects on other, normal cells of a treated patient. Preferably, the methods of the invention are carried out during or after IR treatment, to determine whether additional IR treatment, with or without a radiosensitizer, or another type of therapy altogether, should be carried out. Alternatively, the methods can be carried out before IR treatment, or after some other type of treatment, to see if IR treatment, with or without a radiosensitizer, should be carried out and to provide an indication as to the outcome of such therapy. As is noted above, the methods of the invention can be used to determine specific types, dosages, routes, and schedules of such treatments.

Examples of additional therapies that can be indicated include chemotherapy, biological therapy, gene therapy, oncolytic viral therapy, small molecule therapy, antisense therapy, and therapy involving the use of angiogenesis inhibitors (e.g., angiostatin, endostatin, and icon). Selection of any of these types of therapies, based on gene expression patterns detected using the methods of the invention, can readily be carried out by those of skill in the art.

Specific examples of anticancer agents (i.e., chemotherapeutic agents) that can be selected using the methods of the invention are provided as follows. These compounds fall into several different categories, including, for example, alkylating agents, antineoplastic antibiotics, antimetabolites, and natural source derivatives. Examples of alkylating agents that can be selected using the methods of the invention include busulfan, caroplatin, carmustine, chlorambucil, cisplatin, cyclophosphamide (i.e., cytoxan), dacarbazine, ifosfamide, lomustine, mecholarethamine, melphalan, procarbazine, streptozocin, and thiotepa; examples of antineoplastic antibiotics include bleomycin, dactinomycin, daunorubicin, doxolubicin, idarubicin, mitomycin (e.g., mitomycin C), mitoxantrone, pentostatin, and plicamycin; examples of antimetabolites include fluorodeoxyuridine, cladribine, cytarabine, floxuridine, fludarabine, flurouracil (e.g., 5-fluorouracil (5FU)), gemcitabine, hydroxyurea, mercaptopurine, methotrexate, and thioguanine; and examples of natural source derivatives include docetaxel, etoposide, irinotecan, paclitaxel, teniposide, topotecan, vinblastine, vincristine, vinorelbine, taxol, prednisone, tamoxifen, asparaginase, and mitotane.

The biological therapy that can be selected using the methods of the invention can involve administration of an immunomodulatory molecule, such as a molecule selected from the group consisting of tumor antigens, antibodies, cytokines (e.g., interleukins, interferons, tumor necrosis factor, granulocyte macrophage colony stimulating factor, macrophage colony stimulating factor, and granulocyte colony stimulating factor), chemokines, complement components, complement component receptors, immune system accessory molecules, adhesion molecules, and adhesion molecule receptors.

Oncolytic viral therapy can be selected using the methods of the invention, and can involve the use of, for example, mutant herpes viruses, including or lacking exogenous genes encoding therapeutic molecules. Mutant viruses that can be used in the invention can be derived from members of the family Herpesviridae (e.g., HSV-1, HSV-2, VZV, CMV, EBV, HHV-6, HHV-7, and HHV-8). Specific examples of attenuated HSV mutants that can be used in the invention include G207 (Yazaki et al., Cancer Res. 55(21):4752-4756, 1995), HF (ATCC VR-260), MacIntyre (ATCC VR-539), MP (ATCC VR-735); HSV-2 strains G (ATCC VR-724) and MS (ATCC VR-540), as well as mutants having mutations in one or more of the following genes: the immediate early genes ICP0, ICP22, and ICP47 (U.S. Pat. No. 5,658,724); the γ34.5 gene; the ribonucleotide reductase gene; and the VP16 gene (i.e., Vmw65, WO 91/02788; WO 96/04395; WO 96/04394). The vectors described in U.S. Pat. Nos. 6,106,826 and 6,139,834 can also be used. A time period during which to administer such therapy can be selected based on, for example, detection of increased expression of a gene product that is conducive to the efficacy of oncolytic viral therapy. For example, detection of induction of expression of cellular ribonucleotide reductase, which is useful in viral replication, can be used to indicate an optimal time frame and dosage for use of viral therapy.

Selection of any of these and other types of therapy, based on the analysis of gene expression levels using the methods of the invention, can be carried out by those of skill in this art. Moreover, the design of approaches in which any of these therapies is combined with one another and/or IR therapy is facilitated by the methods of the invention. In addition, as is noted above, information obtained by analysis of a particular class of tumors from one or more subjects can be used in the selection of treatment for another subject having a tumor of that class. For example, if a treatment regimen has been identified for a particular type of tumor, using the methods described herein, that regimen can be used to treat that type of tumor in other patients that have not had gene expression patterns of their tumor or tumors analyzed. This can be done when a tumor in a subject is determined by, for example, histological methods, to be of the same type as a tumor from another subject for which a treatment regimen has already been determined. Similarly, if a subject has more than one of the same type of tumor, it is not necessary to carry out the methods described herein on each separate tumor.

The invention can also be used to identify additional genes that are indicative of sensitivity or resistance to different types of cancer therapy, as well as for characterization of the patterns (e.g., temporal patterns) of expression of these genes in response to different types or amounts of treatment. These genes can be identified using the arrays described above. For example, when it is observed that expression patterns of a particular gene that had not previously been associated with sensitivity or resistance to a particular type of treatment can be correlated with such features, then the gene can be then used as an indicator gene in the methods described above.

The invention also includes kits that can be used in the methods described above. These kits can include nucleic acid molecule arrays, such as those described above, as well as instructions for using the kits to characterize expression patterns of certain genes in cancer cells, leading to the selection of an appropriate treatment strategy, as is discussed above.

Experimental Results and Methods

Summary

U87 cells derived from human malignant gliomas and growth-arrested human embryonic lung (HEL) fibroblasts were examined for their response to ionizing radiation by profiling their RNAs on DNA arrays. In the first series of experiments, cells grown in vitro were harvested and the RNAs were extracted 5 hours after exposure to 1, 3, or 10 Gy. In the second series of experiments, the U87 tumors were implanted in the mice and subjected to the same doses of irradiation. The xenografts were harvested at 1, 5, or 24 hours after irradiation and subjected to the same analyses. We observed and report (i) cell-type common and cell-type specific responses, (ii) genes induced at low levels of irradiation but not at higher doses, (iii) temporal patterns of gene response in U87 xenografts that varied depending on radiation dose and temporal patterns of response that were similar at all doses tested, (iv) significantly higher up-regulation of cells in xenografts than in in vitro cultures, and (v) genes highly up-regulated by radiation. The responding genes could be grouped into 9 functional clusters. The representation of the 9 clusters was to some extent dependent on dose and time post-irradiation. The results show that clinical outcome of ionizing radiation treatment can benefit significantly by taking into account both cell-type and radiation dose specificity of cellular responses.

Materials and Methods

Cell Lines, Animals, and Irradiation

A human malignant glioblastoma cell line, U87, was maintained as described elsewhere (Kataoka et al., Int. J. Radiat. Biol. 76:633-639, 2000), and human embryonic lung (HEL) fibroblasts were also maintained as described elsewhere (Van Sant et al., Proc. Natl. Acad. Sci. U.S.A. 96:8184-8189, 1999). Cells were grown to confluence, maintained in the same medium for two additional days, and irradiated with doses of 1, 3, or 10 Gy using a GE Maxitron Generator operating at 250 kV, 26 mA at a dose rate of 118 cGy/minute. Samples were collected 5 hours after irradiation. U87 Xenografts were transplanted in athymic nude mice, and were irradiated at the same doses, as described elsewhere (Bradley et al., Clin. Cancer Res. 5:1517-1522, 1999). Xenografts were harvested, frozen in liquid nitrogen, and stored at −80° C.

Preparation of Radiolabeled cDNA Probes and Hybridization with DNA Arrays

Total RNA was purified as described elsewhere (Khodarev et al., Proc. Natl. Acad. Sci. U.S.A. 96:12062-12067, 1999). cDNAs were prepared with MMLV reverse transcriptase (GIBCO BRL) in the presence of oligo-dT and γ[³³P]-ATP according to the protocol supplied by Research Genetics (Huntsville, Ala.).

Experimental Design, Data Acquisition, and Analysis

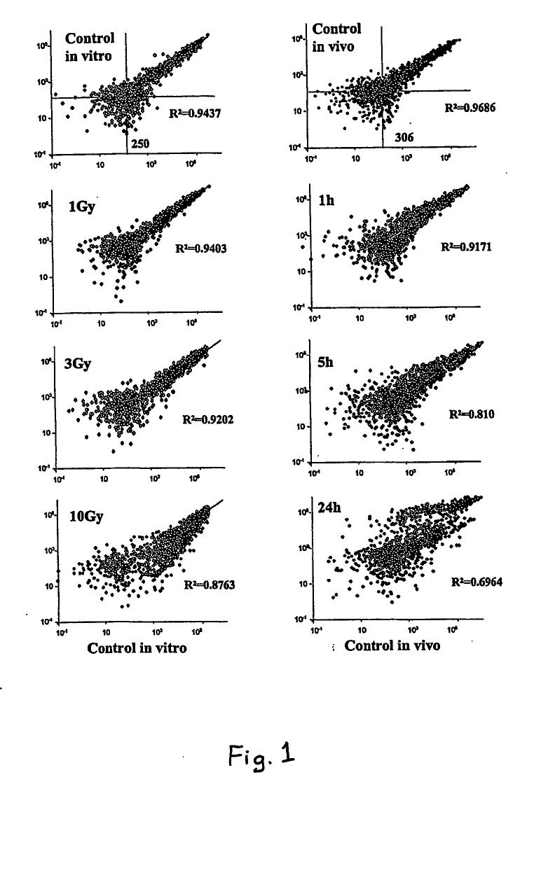

The analyses described herein are based on hybridization data from 48 GeneFilters® GF211 cDNA arrays (Research Genetics). Each GeneFilters® microarray consists of 5,184 distinct sequence-verified probes spotted onto a 5×7 cm positively charged nylon membrane. 4132 spots correspond to unique human genes. The experiments led to the acquisition of approximately 200,000 data points. The experiments on the response of cells grown in cell culture were done in triplicate, with purification of independent RNA samples and independent hybridizations. Quality control of hybridizations was based on internal double-spotted controls for assessment of uniformity of hybridization, estimation of reproducibility assessed by hybridization of the same sample of RNA with two different arrays (see FIG. 1 and below), and estimation of sensitivity and specificity of data acquisition by comparison with visual readings of arrays as described below. The in vivo studies were done on two independent groups of animals randomized by size of the tumor. Each dose of ionizing radiation (1, 3, or 10 Gy) and each time point (1, 5, or 24 hours) were represented by two animals, and cDNA prepared from each xenograft was hybridized independently.

The software package PATHWAYS 2.01®, provided by the manufacturer for acquisition and analysis of GeneFilters® GF211 data, generated many false-positives, especially for low intensity signals. To overcome this problem, numerical signal intensity values for each hybridization spot were determined in a Storm 860 phosphorimager, with the aid of ImageQuant® (Molecular Dynamics).

Data Filtration

Exported intensities of control (un-irradiated) and experimental samples were further filtered, based on the following rules:

(i) Negative values resulting from subtraction of background were transformed to zeros. Data points with zero values in either control or experimental arrays were removed from these analyses.

(ii) The intensity values in each array were normalized with respect to the average intensity value of that array (Freeman et al., Biotechniques 29:1042-1046, 2000).

(iii) All values less or equal to 10% of average intensity (global mean) were transformed to zero. This cut-off value of intensity corresponds to 95.5% of specificity, calculated as true negatives/(true negatives+false positives). Estimation of numbers of true negative, true positive, and false positive data for each cut off value was based on visual examination of array images by experienced readers.

(iv) Estimation of significant levels of response was based on scatterplots of two independent control samples vs. each other and vs. each experimental sample (FIG. 1). For our cell culture data sets, mean+/−one Sd corresponded to 1.45 ratio and 95% confidence interval corresponded to 1.90 ratio. For in vivo data sets, the values increased to 1.54 and 2.08, respectively. We arbitrarily chose ratios of +/−1.60 as cut-off values for matches of independent experiments and comparison of in vitro and in vivo data. For comparison of independent experiments and for statistical analysis of data, we used JMP software (SAS Institute Inc., Cary, N.C.). The identified genes that responded in more than one cell line in cultured cells or both in culture and in xenografts were obtained using the “join” function of the JMP software. For data clustering, we used the hierarchical clustering option, provided by the JMP software

Selected genes were checked using BLAST and Human Genome Browser (http://genome.ucsc.edu/goldenPath/.) Annotations of genes were based on PubMed, OMIM, and other databases.

Experimental Results

Experimental Design

We report two series of experiments. In the first series, we irradiated confluent cultures of U87 human malignant glioblastoma cells or of HEL fibroblasts with 1, 3, or 10 Gy. The cells were harvested at 5 hours after irradiation, and total RNA was processed and analyzed as described above in Materials and Methods. The identification of genes that are up- or down-regulated as a consequence of irradiation was based on reproducibility of experimental results in independent experiments as described in Materials and Methods. The key parameters of the study were as follows: of the 4132 genes represented in the cDNA arrays, the average number of genes having transcripts that were detected were 1858 for mock-treated U87 cells and 1973 for HEL fibroblasts. Of this number, those with b values comprising at least 90% of the mean value and which were included in these analyses were 1470 and 1591, respectively. The corresponding number of genes included in these analysis for 1, 3, or 10 Gy were 1494, 1374, and 1318, respectively, for U87 cells and 1495, 1507, and 1609, respectively, for HEL fibroblasts.

In the second series of experiments, we irradiated U87 implants in the hind limb of mice as described in Materials and Methods. In this instance, the mice received 1, 3, or 10 Gy and xenografts were removed and processed at 1, 5, or 24 hours after mock-treatment or irradiation. In this series of experiments, we detected on the average 1973 transcripts from mock-treated tumors. Of this number, those with intensity values above the cut off value (see Methods, above) and which were included in these analyses were 1591. The corresponding average number of genes included in these analysis for 1, 3, or 10 Gy were 1274, 1303, and 1244, respectively, for U87 cells and 1495, 1507, and 1609, respectively, for HEL fibroblasts.

Analyses of Irradiation Dose-Dependent Responders in U87 and HEL Cell Cultures

The responders were analyzed with respect to two criteria. The first compared the overall kinetics of up regulation as a function of dose. In Table 1, the first three columns show all possible permutations of up-regulated (+) genes. The remarkable aspects of the data are the large number of genes that were up-regulated to their highest levels after either 1 Gy, 3 Gy, or 10 Gy. Only a small number of genes were up-regulated to their highest level after 1 Gy and remained at the same level in cell exposed to 3 or 10 Gy. The overall impression is that more genes are transcriptionally activated as a function of dose than those having a transcript amount that increased after 1 Gy and then declined at higher doses.

The second criterion for the analyses was the identification of responders common to both U87 and HEL cells. The results summarized in Table 2 show that the shared responders were 10 for 1 Gy, 11 for 3 Gy, and 48 for 10 Gy. The results indicate that the responders represented three groups: those that were U87 cell-specific, those that were HEL fibroblast-specific, and those that were both U87- and HEL fibroblast-specific. Although the numbers were small, the bulk of the shared genes were identified in cells exposed to 10 Gy, consistent with the data showing that 10 Gy induced the highest number of responders.

Analyses of Responders to Irradiation of Implanted Xenografts of U87 Cells

The results of the analyses carried out on responders to irradiation of xenografts of the U87 cells are shown in Table 2. There were 542 responders to 1 Gy, 554 responders to 3 Gy, and 536 responders to 10 Gy. Of this number, the 4 ^{th} column of the table lists number of responders in U87 cells grown in vitro and those in the transplanted xenografts. These numbers, 25 at 1 Gy, 25 at 3 Gy, and 42 at 10 Gy, reflect the small number of responders in cultured cells in vitro. It is noteworthy, however, that the responders common to irradiated cultured cells and xenografts represent >20 percent of the genes that are up-regulated following irradiation of U87 cultured cells. Analyses of the results showed that 15 genes were up-regulated by ionizing irradiation of HEL fibroblasts and of the U87 cells cultured in vitro and in xenografts. These are identified in Table 3.

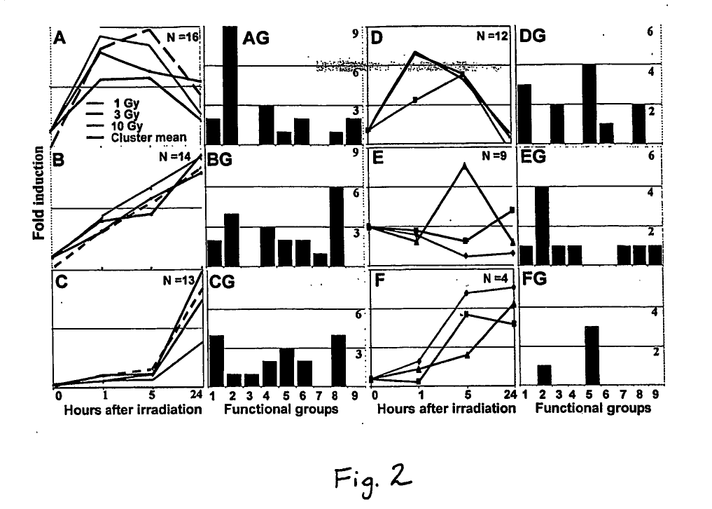

Earlier, we showed that analyses of the effects of exposure to ionizing radiation between 1 and 10 Gy of cells in culture can be either dose dependent or independent. The U87 genes of cells grown in vitro or in transplanted xenografts and up-regulated by ionizing radiation were analyzed for dependence on radiation dose and time of response. The results shown in FIG. 2, panels A-F, are as follows: panels A-D illustrate a subset of genes having temporal responses to irradiation that were for the most part radiation dose-independent. Thus, the general pattern of response for each gene in these clusters was similar, if not identical, at 1, 3, and 10 Gy. Panels E and F of FIG. 1 illustrate genes having temporal responses to radiation that were dose dependent. The number of genes in each cluster (N) is indicated in each panel. The genes illustrated in FIG. 1 are identified in the Table 3.

The Function of Genus Up-Regulated by Radiation

We used a modified functional classification, suggested by Stanton et al. (Circ. Res. 86:939-945, 2000), to characterize the genes in our analysis. These groups are genes involved in: (1) cell/organism defense and homeostasis; (2) cell-cell interactions and cell signaling; (3) cell cytoskeleton/motility/ECM; (4) RNA transcription processing/transport; (5) protein synthesis/modifications/transport; (6) metabolism/mitochondrion; (7) DNA metabolism/chromatin structure; (8) oxidative stress/apoptosis; and (9) unclassified. The distribution of responders by functional groups is shown in Table 3. A more restricted distribution based on a total of 68 genes is shown in FIG. 2, Panels AG to FG. The significant observation to come out of these analyses is that genes involved in cell-cell communication and signaling appear to be induced at relatively low IR levels. In contrast, genes involved in oxidative stress and apoptosis are more likely to be induced after irradiation with 3 or 10 Gy. Several groups were underrepresented, but this may be due to the number of genes belonging to that group and which were included in the cDNA arrays.

The results of additional experiments carried out using U87 xenografts implanted into athymic nude mice are provided in Table 4. These experiments were carried out using different amounts of radiation (1, 3, or 10 Gy), and the data were obtained after different lengths of time after treatment (1, 5, or 24 hours), as indicated. As is shown in the Table, we found that the expression of numerous genes was affected by the different amounts of treatments, and that the level of expression varied at the different times after treatments.

As is noted above, interaction of the Fas ligand with the Fas receptor (Apo-l, CD-95) induces apoptotic cell death. In further experiments, we found that radiation-induced temporal transcriptional changes of the FAS receptor coincide with FAS ligand induced cytotoxicity (see FIG. 3). In particular, HUVE cells were treated with ionizing radiation (9 Gy), and the pattern of expression of FAS receptor mRNA in these cells after radiation treatment was observed for 24 hours. When these cells were also contacted with an anti-FAS antibody, the number of apoptotic cells present increased in a manner that paralleled the increase in transcription of the FAS receptor. These results show that analysis of the expression of a gene after a particular treatment (e.g., ionizing radiation) can be used to identify an optimal time frame during which to administer a treatment. This enables limiting the time during which a patient is exposed to the treatment, without loss of significant therapeutic benefit.

Discussion of Experimental Results

We have identified 3 sets of genes that are activated by ionizing radiation. The first set is shared by HEL fibroblasts and U87 malignant glioma cells that are grown in culture and harvested 4 hours after irradiation with 1, 3, or 10 Gy. The second set is shared between U87 cells grown in vitro and those transplanted as xenografts in the hind limb of mice. The last and the smallest group are 15 genes induced in all irradiated cells, whether grown in vitro or in mouse xenografts. We also characterized the expression of numerous additional genes in U87 xenografts. The significance of certain aspects of the data are discussed as follows:

(i) The response to IR consists of elements that are both cell common and cell-type specific.

(ii) Within the lethal range of IR administration, the response of a significant number of genes was dose dependent. As is illustrated herein in part in Table 1 and in Panels A-D of FIG. 1, some genes were induced at low IR doses and some only at high IR doses. This finding is in conflict with the previously prevailing notion in the art that, within certain parameters, the sum total, rather than individual doses, predicts success of IR treatment.

(iii) Another finding of considerable interest is that, in several instances, the temporal pattern of gene expression was also dose dependent. The brief expression of certain genes can play a significant role in determining whether the cell survives or dies following irradiation. Also, detection of these brief periods of expression indicates precise time periods during which a particular treatment, related to the observed expression, can be given.

The present studies have identified several genes of particular interest that are inducible by IR A few genes induced by IR in multiple systems analyzed in this study appear at first glance to be of particular interest. They are as follows:

(i) β2-microglobulin is a common radiation responder (Table 3). Intracellular assembly of MHC class I heavy chains with β2-microglobulin occurs prior to the expression of the antigen-presenting complex on the cell surface. Treatment of human β2-microglobulin (β2m) with hydroxyl radicals generated by treatment with gamma-radiation resulted in the disappearance of the Mᵣ12,000 protein and the appearance of a cross-linked complex stable under reducing conditions and in sodium dodecyl sulfate (Capeillere-Blandin et al., Biochem J. 277:175-182, 1991). Augmentation of MHCI/β2m complexes by increasing doses of irradiation has been observed in short-term cultures, established from eight human glioblastomas (Klein et al., J. Neurosurg. 80:1074-1077, 1994). Both MHC class I and β2-microglobulin genes were activated in the systems tested in this study. One hypothesis that could explain these results is accelerated degradation of damaged or misfolded proteins caused by IR.

(ii) Protein phosphatase 2A (PP2A) down-regulates the mitogen-activated protein kinase (MAPK) cascade, relays signals for cell proliferation, and appears to be linked to carcinogenesis. The PP2A holoenzyme exists in several trimeric forms, consisting of a Mᵣ 36,000 PP2A-C core catalytic subunit; a Mᵣ 65,000 structural/regulatory component, PP2A-A; and a variable regulatory subunit, PP2A-B, which confers distinct properties on the holoenzyme. Each subunit exists in multiple isoforms, encoded by different genes. Consequently, the PP2A trimer exists in many different configurations, which differ in expression patterns and specificity. The gene identified at 11q23 (Wang et al., Science 282:284-287, 1998) and designated PPP2R1B encodes the structural-regulatory A subunit PP2A-A-β. This subunit is required for the interaction of the catalytic PP2A-C and variable PP2A-B subunits and is critical for phosphatase activity. Recently it has been shown that PP2A is required for regulation of DNA-PK (Douglas et al., J. Biol. Chem. 276:18992-18998, 2001). DNA-dependent protein kinase (DNA-PK) is a complex of DNA-PK catalytic subunit (DNA-PKcs) and the DNA end-binding Ku70/Ku80 heterodimer. DNA-PK is required for DNA double strand break repair by the process of nonhomologous end joining. Nonhomologous end joining is a major mechanism for the repair of DNA double strand breaks in mammalian cells. As such, DNA-PK plays essential roles in the cellular response to ionizing radiation and in V(D)J recombination. In vitro, DNA-PK phosphorylation of all three protein subunits (DNA-PK catalytic subunit, Ku70, and Ku80) inactivation of the serine/threonine protein kinase activity of DNA-PK. Phosphorylation-induced loss of the protein kinase activity of DNA-PK was restored by the addition of the purified catalytic subunit of either protein phosphatase 1 or PP2A. Reversible protein phosphorylation is an important mechanism for the regulation of DNA-PK protein kinase activity and that the protein phosphatase responsible for reactivation in vivo is a PP2A-like.

(iii) Unexpected is the upregulation by IR of several genes classified in the RNA splicing/nuclear cytoplasmic RNA transport functional group. Two genes, the survival of motor neuron (SMN) interacting protein 1 (SIP-1 or Gemin 2) and U1 snRNP70 genes, both belong to cluster A (FIG. 1 and Table 3). SIP-1 interacts with SMN and is involved in the assembly/metabolism of snRNPs, as well as in their nuclear-cytoplasmic transport (Wang et al., J. Biol. Chem. 276:9599-9605, 2001). Also, RNPS1, in cluster C (FIG. 1 and Table 3) is a general activator of pre-mRNA splicing (Mayeda et al., EMBO J. 18:4560-4570, 1999). In addition, both hnRNPA1 and hnRNPE2 are up-regulated following IR in both U87 and HEL cell lines (Table 3). hnRNPs mediate several RNA-related functions, including pre-mRNA splicing and mature mRNA transport to cytoplasm. hnRNPA1 was recently isolated among 12 other hypoxia-responsive genes from cervical cancer cells, and proteomics analyses identified RNA-binding motif-containing proteins, mostly involved in RNA splicing, as major caspase-3 targets during the Fas-induced apoptosis in T cells (Thiede et al., J. Biol. Chem. 276:26044-26050, 2001). These data show that pathways of nuclear pre-mRNA processing and nuclear/cytoplasmic transport of RNA are activated by IR, providing additional therapeutic targets.

(iv) Transcriptional activation of actin genes by IR was reported by Woloschak et al. (Int. J. Radiat. Biol. 59:1173-1183, 1991). The results reported here indicate that actin α2 and β-actin were induced in all cells subjected to IR and were co-clustered (see FIG. 1, cluster D and Table 3). These genes are frequently classified as housekeeping genes that are expressed in mock-treated and stressed cells. A more likely explanation consistent with other data is that different components of the cytoskeleton are specifically involved in the stress response and are transcriptionally controlled through p53-dependent mechanisms (Zhao et al., Genes Dev. 14:981-993, 2000).

(v) Cyp33 belongs to the cluster C, which includes the most highly up-regulated in vivo genes. The Mᵣ 33,000 CYP33 protein exhibits RNA-binding, peptidylprolyl cis-trans isomerase, and protein folding activities. CYP33 is the first example of a protein that combines RNA-binding and PPIase activities. An identical transcript was detected in a small cell lung cancer (SCLC) cell line (Kim et al., Oncogene 17:1019-1026, 1998). Recent reports indicate that Cyp33 is involved in regulation of MLL1 (Mixed Lineage Leukemia 1) (Fair et al., Mol. Cell Biol. 21:3589-3597, 2001). Overexpression of the Cyp33 protein in leukemia cells results in altered expression of HOX genes that are targets for regulation by MLL. These alterations are suppressed by cyclosporine and are not observed in cell lines that express a mutant MLL protein. These results suggest that binding of Cyp33 to MLL modulates its effects on the expression of target genes.

(vi) Several genes associated with the endoplasmic reticulum and secretory pathways were up-regulated by IR (calumenin, golgin-95, and LDLC) (see Table 3). Members of the CREC family localize to the secretory pathway of mammalian cell and include reticulocalbin, ERC-55/TCBP-49/E6BP, Cab45, calumenin, and crocalbin/CBP-50 (Klein et al., J. Neurosurg. 80:1074-1077, 1994). Calumenin, a calcium binding protein, is related to the CREC family of proteins. Recent reports indicate that some CREC family members are involved in pathological activities such as malignant cell transformation, mediation of the toxic effects of snake venom toxins, and putative participation in amyloid formation.

TABLE 1
Number of genes up- or down-regulated after irradiation
of U87 or and HEL cells in culture.

| | Number of genes^{a} | |
|---|---|---|
| Dose (Gy) | U87 | HEL |

| 1 | 3 | 10 | ↑ | ↓ | ↑ | ↓ |
|---|---|---|---|---|---|---|
| + | − | − | 9 | 26 | 2 | 40 |
| + | + | − | 6 | 2 | 0 | 9 |
| + | − | + | 16 | 8 | 9 | 12 |
| + | + | + | 6 | 3 | 6 | 7 |
| − | + | + | 9 | 18 | 14 | 18 |
| − | + | − | 11 | 10 | 9 | 16 |
| − | − | + | 32 | 132 | 85 | 90 |

^{a}+, responders;
−, nonresponders;
↑, up-regulated;
↓, down-regulated.

TABLE 2
Summary of responders to ionizing radiation in U87 cell grown
in cell culture (U87-C) or transplated in xenografts (U87-X)
or in human embryonic lung cells grown in culture (HEL-C)^{a}

| | | | | | U87-HEL | | | | | U87-C/X | | | All cells |
|---|---|---|---|---|---|---|---|---|---|---|---|---|---|
| | U87-C | | HEL-C | | common | | | U87-X | | common | | | common |
| Gy | ↑ | ↓ | ↑ | ↓ | ↑ | ↓ | ↑↓ | ↑ | ↓ | ↑ | ↓ | ↑↓ | ↑ |
| 1 | 37 | 39 | 17 | 68 | 6 | 2 | 2 | 481 | 61 | 17 | 0 | 10 | 1 |
| 3 | 32 | 33 | 28 | 48 | 5 | 4 | 2 | 533 | 21 | 18 | 1 | 7 | 3 |
| 10 | 63 | 161 | 114 | 125 | 14 | 12 | 22 | 532 | 4 | 26 | 1 | 0 | 4 |

^{a}↑ - up-regulated,
↓ - down-regulated.
↑↓ - genes up-regulated in one cell line but down-regulated in another.

TABLE 3
Common genes, responding to irradiation in U87 and HEL cell lines and U87 xenografts

| | | U87-C | | | HEL-C | | | U87-X | | | |
|---|---|---|---|---|---|---|---|---|---|---|---|
| ACC | Group and name | 1 Gy | 3 Gy | 10 Gy | 1 Gy | 3 Gy | 10 Gy | 1 Gy | 3 Gy | 10 Gy | Comments |

I. Homeostasis/self defense

| | | | | | | | | | | | |
|---|---|---|---|---|---|---|---|---|---|---|---|
| AA464246 | HLA-C | 1.28 | 1.20 | 0.51 | 0.98 | 0.91 | 2.65 | 1.71 | 1.74 | 1.53 | ↓ D |
| AA34117 | HLA-B-assot. G9a | 0.89 | 0.57 | 0.51 | 0.72 | 0.78 | 0.61 | | | | $ |
| AA670408 | β2-microglobulin | 1.02 | 2.14 | 0.53 | 1.43 | 1.82 | 5.78 | 1.80 | 1.93 | 1.24 | $ * D |
| AA778663 | 4-1BB ligand | 1.67 | 1.38 | 0.35 | | | | 4.43 | 10.70 | 12.1 | * A |
| AA136271 | CD58 (LFA3) | 1.06 | 0.89 | 0.20 | 0.51 | 0.90 | 0.59 | | | | $ |
| R77293 | ICAM1 | 1.22 | 0.99 | 0.42 | | | | 2.00 | 1.47 | 2.46 | ↓ B |
| AA130584 | CEACAM5 | 1.77 | 1.28 | 1.88 | | | | 3.22 | 18.63 | 3.64 | * A |
| N51018 | Biglycan | 1.33 | 1.22 | 1.64 | | | | 0.47 | 0.74 | 1.69 | * E |
| AA399674 | SPRR2C | 0.85 | 0.89 | 0.43 | 0.62 | 0.84 | 1.79 | | | | ↓ |
| T49657 | K+ channel TASK | 2.09 | 1.61 | 2.36 | 1.60 | 1.89 | 1.68 | 4.62 | 3.90 | 5.36 | S * A |
| AA069770 | K+ channel KCNB1 | 0.56 | 1.53 | 1.59 | | | | 10.07 | 7.12 | 6.79 | ↓ C |
| H14808 | Na+/K+ ATPase β 2 | 1.46 | 0.93 | 2.11 | | | | 3.07 | 2.43 | 6.45 | * A |
| H24316 | aquaporin | 1.03 | 1.63 | 1.44 | | | | 24.56 | 12.25 | 5.84 | * C |
| H57136 | PLM CTchannel | 1.23 | 1.80 | 1.60 | 0.87 | 0.84 | 0.53 | 2.72 | 4.17 | 4.44 | ↓ * C |
| AA402891 | transporter ENT2 | 1.08 | 1.19 | 1.60 | | | | 6.07 | 36.08 | 2.61 | * C |
| AA191488 | Cu2 + uptake protein CTR1 | 1.72 | 1.00 | 2.46 | 2.42 | 3.20 | 4.39 | 1.05 | 1.27 | 1.80 | $ * E |
| AA480459 | Transcobalamin II | 0.76 | 0.61 | 0.55 | 0.47 | 0.83 | 0.31 | | | | $ |
| H72723 | MT1B | 1.12 | 0.61 | 0.57 | | | | 2.04 | 2.93 | 5.46 | ↓ D |
| H23187 | Carbonic anhydrase II | 0.84 | 1.10 | 0.59 | | | | 4.41 | 2.50 | 2.26 | ↓ B |

II. Cell-cell communications/signaling

| | | | | | | | | | | | |
|---|---|---|---|---|---|---|---|---|---|---|---|
| R56211 | PDGFRβ | 1.02 | 1.12 | 1.87 | 0.92 | 1.37 | 1.85 | | | | $ |
| AA486393 | IL 10 receptor β | 1.72 | 1.19 | 1.60 | | | | 6.12 | 3.84 | 3.78 | * A |
| AA485226 | Vitamin D receptor | 2.31 | 0.69 | 0.64 | | | | 6.54 | 9.35 | 3.34 | * A |
| H54023 | MIR-10 (LILRB2) | 1.71 | 1.00 | 1.35 | | | | 0.48 | 0.63 | 1.98 | ↓ E |
| AA400973 | lipocalin 2 | 1.16 | 0.56 | 0.49 | 0.68 | 0.62 | 0.53 | | | | $ |
| AA485922 | copine I | 1.65 | 1.06 | 1.08 | | | | 3.84 | 3.74 | 4.24 | * A |
| R73545 | Flotillin 2 | 1.18 | 0.94 | 0.61 | 1.11 | 1.17 | 1.89 | | | | ↓ |
| N20203 | BMP receptor II | 0.60 | 0.79 | 0.97 | | | | 5.54 | 2.88 | 2.00 | ↓ B |
| AA450062 | BMP, placental | 1.45 | 1.49 | 1.60 | 1.35 | 0.82 | 1.62 | | | | $ |
| AA489383 | BMP 2 | 1.71 | 1.61 | 1.80 | 0.61 | 2.14 | 1.61 | 1.77 | 1.69 | 1.06 | $ * F |
| T55558 | CSF 1 | 0.92 | 1.29 | 0.56 | 0.83 | 0.61 | 0.52 | | | | $ |
| AA486072 | RANTES | 1.14 | 0.67 | 0.42 | | | | 4.92 | 2.79 | 3.74 | ↓ A |
| R43320 | G-protein GNAO1 | 1.79 | 1.05 | 1.83 | | | | 0.61 | 0.82 | 1.65 | E |
| R56046 | G-protein GNAZ | 0.74 | 0.87 | 0.49 | 0.85 | 0.93 | 1.77 | 0.73 | 0.67 | 1.75 | ↓ E |
| AA458785 | guanylate cyclase β1 | 1.74 | 1.45 | 1.86 | | | | 3.89 | 3.75 | 4.07 | *A |
| R37953 | adenylyl cyclase | 1.31 | 1.23 | 0.59 | 1.04 | 1.04 | 2.60 | | | | ↓ |
| | associated protein | | | | | | | | | | ↓ |
| N28497 | PP2A (PPP2R1B) | 1.16 | 1.35 | 2.00 | 0.78 | 0.77 | 0.33 | 7.81 | 6.84 | 5.31 | ↓ * B |
| H15718 | protein kinase AXL | 0.76 | 0.59 | 0.80 | 0.48 | 0.57 | 0.52 | | | | $ |
| AA453789 | protein kinase 7 | 0.94 | 1.19 | 0.60 | 0.69 | 1.04 | 0.53 | | | | $ |
| R59598 | protein kinase Syk | 0.56 | 0.64 | 0.48 | 0.54 | 0.61 | 0.76 | | | | $ |
| R80779 | protein kinase MLK-3 | 1.15 | 1.50 | 1.66 | | | | 10.99 | 6.73 | 4.43 | * B |
| AA890663 | protein kinase PAK1 | 0.64 | 1.95 | 1.80 | 0.90 | 1.06 | 0.56 | | | | ↓ |
| N52958 | SLP-76 | 1.38 | 2.21 | 1.72 | | | | 1.07 | 1.32 | 1.80 | * |
| H73724 | CDK6 | 1.76 | 1.53 | 2.08 | | | | 0.47 | 0.81 | 2.01 | * E |
| AA464731 | calgizzarin | 1.01 | 0.96 | 0.33 | 1.06 | 1.07 | 2.35 | | | | ↓ |
| N63940 | Acetylholinesterase | 0.83 | 0.61 | 0.87 | 0.65 | 0.62 | 0.84 | | | | $ |

III. Cytoskeleton/motility

| | | | | | | | | | | | |
|---|---|---|---|---|---|---|---|---|---|---|---|
| AA703141 | protein 4.1 (EBP41) | 0.60 | 0.63 | 0.64 | 0.61 | 0.81 | 1.34 | | | | $ |
| AA877166 | Myosin light chain 2 | 0.83 | 1.01 | 0.57 | 0.83 | 0.86 | 0.49 | | | | $ |
| AA504625 | kinesin heavy chain | 0.62 | 1.34 | 0.59 | | | | 4.42 | 12.29 | 0.25 | * C |
| AA868929 | Troponin T1 | 1.01 | 0.53 | 0.27 | | | | 0.18 | 0.12 | 1.98 | * |
| R44290 | β-actin (ACTB) | 1.13 | 0.60 | 0.55 | 1.32 | 1.47 | 3.48 | 1.79 | 1.93 | 1.43 | ↓ D |
| AA634006 | Actin α-2 (ACTSA) | 1.51 | 0.80 | 0.51 | 1.01 | 1.30 | 3.16 | 1.55 | 1.55 | 1.52 | ↓ D |
| AA629189 | Keratin 4 (KRT4) | 1.08 | 1.08 | 1.62 | 1.02 | 1.32 | 0.39 | | | | ↓ |

IV. RNA synthesis/modifications

| | | | | | | | | | | | |
|---|---|---|---|---|---|---|---|---|---|---|---|
| H99588 | LAF4 | 3.15 | 1.23 | 2.62 | 3.62 | 2.70 | 4.83 | 0.59 | 0.82 | 1.65 | $ * E |
| N47099 | SMAD2 | 1.12 | 1.64 | 2.04 | 0.94 | 1.90 | 0.55 | | | | $ |
| AA478268 | CTBP 1 | 1.17 | 1.63 | 1.44 | | | | 3.99 | 5.34 | 2.77 | * B |
| AA394127 | NF-AT3 | 1.24 | 0.96 | 0.36 | 1.15 | 0.90 | 2.19 | | | | ↓ |
| AA258001 | RELB | 1.02 | 1.10 | 0.59 | | | | 5.12 | 7.57 | 2.33 | ↓ C |
| AA253434 | transcription factor HSF2 | 1.08 | 0.60 | 1.06 | | | | 5.09 | 2.64 | 1.86 | ↓ C |
| AA457155 | ZNF212 | 0.49 | 0.68 | 1.32 | 2.02 | 0.87 | 2.45 | | | | ↓ |
| R02346 | U1 snRNP 70 | 2.57 | 1.32 | 2.59 | 1.33 | 1.51 | 1.98 | 6.29 | 5.46 | 8.60 | $ * A |
| AA496879 | RNP S1 | 1.58 | 1.70 | 2.43 | 1.18 | 1.02 | 0.47 | 7.72 | 7.05 | 4.26 | $ * B |
| N26026 | Gemin2 (SIP1) | 2.31 | 2.94 | 3.88 | | | | 5.00 | 4.38 | 3.32 | * A |
| AA126911 | hnRNP A1 | 0.99 | 0.57 | 0.39 | 1.65 | 1.81 | 4.53 | | | | ↓ |
| AA431440 | hnRNP-E2 | 0.81 | 0.64 | 0.61 | 0.83 | 1.09 | 2.01 | | | | ↓ |
| T60163 | RNase L | 1.13 | 1.09 | 1.64 | | | | 6.84 | 8.45 | 3.32 | * B |

V. Protein synthesis/modifications

| | | | | | | | | | | | |
|---|---|---|---|---|---|---|---|---|---|---|---|
| R43973 | EF1γ | 2.23 | 1.22 | 0.54 | 1.69 | 1.30 | 3.83 | | | | $ |
| R54097 | elF-2b | 0.91 | 0.63 | 0.50 | 0.55 | 0.98 | 2.15 | | | | ↓ |
| AA873351 | RPL35a | 0.97 | 2.30 | 1.61 | 0.93 | 0.91 | 2.28 | 1.91 | 1.67 | 1.34 | $ * D |
| T69468 | RPS4Y | 0.51 | 1.01 | 0.89 | 1.95 | 1.87 | 3.26 | | | | ↓ |
| AA490011 | RPL38 | 1.43 | 0.92 | 0.22 | 0.99 | 1.40 | 2.97 | | | | ↓ |
| T67270 | RPL10 | 1.97 | 1.29 | 0.61 | 1.22 | 1.31 | 2.75 | | | | ↓ |
| AA464743 | RPL21 | 1.09 | 1.31 | 0.52 | 0.87 | 0.91 | 1.73 | | | | ↓ |
| AA680244 | RPL11 | 1.01 | 1.18 | 0.25 | 0.84 | 1.18 | 2.94 | | | | ↓ |
| W96450 | aa-tRNA synthetase FARSL | 1.10 | 1.67 | 1.46 | | | | 1.97 | 1.84 | 1.36 | *F |
| AA599158 | aa-tRNA syntetase EPRS | 0.97 | 0.89 | 0.56 | | | | 2.20 | 1.84 | 1.31 | ↓ F |
| AA664241 | alpha-NAC | 0.60 | 0.83 | 2.07 | 0.89 | 1.09 | 1.68 | | | | $ |
| AA424786 | golgin-95 | 0.90 | 0.99 | 0.57 | | | | 10.02 | 19.14 | 2.95 | ↓ C |
| AA457114 | protein B94 | 1.85 | 1.14 | 1.95 | | | | 4.01 | 2.33 | 6.45 | * D |
| AA504455 | LDLC | 1.71 | 1.92 | 1.62 | | | | 2.43 | 1.88 | 1.50 | * F |
| R78585 | calumenin | 0.99 | 1.02 | 0.53 | 1.02 | 1.26 | 2.89 | 1.06 | 1.44 | 1.14 | ↓ D |
| T71316 | ADP-ribosylation factor 4 | 1.25 | 0.79 | 0.48 | 0.94 | 1.05 | 3.12 | | | | ↓ |
| AA455301 | protein GPAA1 | 0.58 | 0.58 | 0.93 | | | | 2.43 | 2.11 | 1.57 | ↓ B |
| N78843 | CYP-33 (PPIE) | 1.79 | 1.47 | 1.69 | | | | 8.57 | 5.27 | 3.85 | * C |
| H98666 | PCOLN3 | 0.80 | 0.60 | 0.50 | 0.78 | 0.61 | 0.60 | | | | $ |
| AA430524 | ACE | 1.17 | 1.70 | 1.99 | | | | 1.78 | 2.85 | 1.66 | * A |
| AA410517 | Serpin PTI | 0.91 | 1.47 | 1.79 | 1.31 | 1.60 | 2.72 | | | | $ |
| W61361 | Serpin CAP2 | 1.24 | 1.76 | 1.27 | | | | 6.67 | 8.93 | 3.52 | * C |
| AA430512 | Serpin CAP3 | 1.22 | 1.53 | 1.69 | | | | 6.45 | 5.37 | 4.20 | * B |
| AA402874 | protective protein | 1.15 | 0.76 | 0.55 | | | | 1.20 | 1.21 | 1.42 | ↓ D |

VI + VIII. Metabolism/energy/oxidative stress

| | | | | | | | | | | | |
|---|---|---|---|---|---|---|---|---|---|---|---|
| N33331 | PPARδ | 1.15 | 1.62 | 1.45 | | | | 34.35 | 50.10 | 95.30 | * C |
| AA465366 | Leukotriene A4 hydrolase | 1.69 | 2.28 | 0.85 | | | | 4.58 | 7.54 | 3.17 | * C |
| R55046 | MpV17 (peroxisome) | 0.75 | 0.60 | 0.57 | 1.30 | 1.69 | 2.18 | | | | ↓ |
| W49667 | fatty acid desaturase | 1.08 | 0.43 | 1.08 | | | | 4.36 | 4.38 | 3.79 | ↓ A |
| W95082 | (11-β)-hydroxysteroid | 1.07 | 0.80 | 0.45 | 0.45 | 0.82 | 0.44 | 16.25 | 5.88 | 3.48 | $ B |
| | dehydrogenase | | | | | | | | | | |
| T73294 | P-450 reductase | 1.78 | 1.05 | 2.01 | | | | 0.80 | 0.71 | 2.69 | |
| AA708298 | H+ ATP synthase | 1.20 | 1.02 | 0.49 | 1.11 | 1.62 | 5.38 | 1.85 | 1.46 | 1.30 | ↓ D |
| H61243 | Uncoupling protein 2 | 0.93 | 0.82 | 0.53 | | | | 1.07 | 0.93 | 1.81 | ↓ |
| W96179 | Glutamate-cysteine ligase | 2.67 | 2.03 | 3.18 | | | | 6.69 | 4.97 | 4.54 | * A |
| AA463456 | Glutathione synthetase | 0.94 | 0.73 | 0.32 | | | | 1.73 | 0.94 | 1.43 | ↓ |
| AA290738 | GSTM4 | 1.10 | 0.93 | 0.46 | | | | 4.34 | 2.92 | 2.00 | ↓ B |
| R52548 | SOD-1 | 0.92 | 0.84 | 0.60 | | | | 6.58 | 3.01 | 2.12 | ↓ B |
| R39463 | Aldolase C | 0.98 | 0.90 | 0.58 | 0.62 | 0.77 | 0.47 | | | | $ |
| H05914 | LDHA | 0.84 | 1.27 | 0.59 | 1.15 | 1.12 | 2.68 | | | | ↓ |
| AA629567 | HSP73 | 1.07 | 0.86 | 0.49 | 1.00 | 1.39 | 2.40 | | | | ↓ |

VII. DNA metabolism/chromatin structure

| | | | | | | | | | | | |
|---|---|---|---|---|---|---|---|---|---|---|---|
| H15112 | Uracil-DNA glycosylase 1 | 0.45 | 0.58 | 1.64 | 0.85 | 1.36 | 1.70 | | | | $ |
| N26769 | DNA glycosylase (MPG) | 0.89 | 0.94 | 0.55 | | | | 5.88 | 3.06 | 1.70 | ↓ B |
| AA608557 | XPE1 (DDB1) | 1.61 | 1.16 | 1.98 | | | | 0.68 | 0.77 | 1.76 | * E |
| AA035095 | BCR protein 1 | 0.99 | 0.74 | 0.52 | 0.65 | 0.93 | 0.54 | | | | $ |
| AA460927 | translin | 0.53 | 1.29 | 2.49 | 0.99 | 1.51 | 2.37 | | | | $ |
| AA442991 | Prothymosin alpha (PTMA) | 2.56 | 2.01 | 1.06 | 2.19 | 1.61 | 2.66 | | | | $ |
| AA456077 | centromere protein p27 | 0.86 | 0.72 | 0.44 | 0.62 | 0.88 | 0.41 | | | | $ |
| R56871 | chromatin assembly factor-1 | 0.69 | 0.98 | 1.82 | 0.94 | 0.30 | 0.60 | | | | ↓ |

IX. Unclassified

| | | | | | | | | | | | |
|---|---|---|---|---|---|---|---|---|---|---|---|
| AA683321 | PAR-5 | 1.72 | 1.27 | 1.38 | 2.26 | 2.62 | 8.38 | 0.44 | 0.72 | 1.61 | ↓ $ E |
| R06254 | protein D54 | 1.62 | 0.92 | 1.68 | 1.26 | 1.29 | 1.87 | | | | $ |
| AA406064 | BPY1 | 1.78 | 1.86 | 2.12 | | | | 1.68 | 2.57 | 1.02 | * D |
| AA448289 | protein D123 | 1.14 | 1.05 | 2.08 | | | | 9.12 | 10.66 | 9.08 | * C |
| N34095 | FEZ2 | 1.14 | 1.55 | 1.69 | | | | 2.86 | 3.20 | 3.70 | * A |
| R87497 | 2.19 gene | 1.85 | 2.70 | 1.71 | | | | 4.32 | 3.50 | 5.08 | * A |
| AA452826 | Purkinje cell protein 4 | 0.60 | 0.80 | 0.59 | | | | 5.26 | 3.82 | 3.32 | ↓ D |

Legend to Table 3.
Common genes, responding to irradiation in U87/HEL cell lines and/or U87 cell line/U87 xenografts. Shown
are genes that responded to irradiation in both U87 and HEL cell lines in vitro or U87, grown in cell culture
(U87-C) or transplanted in xenografts (U87-X). Genes are distributed according cell functions (see text, p10).
Numbers are average ratios of significant up or down regulation for each dose tested at 5 hours after
irradiation. Symbols in columns 12 and 24 (“Comments”) are:
$ - consistent up or down regulation in both U87 and HEL in vitro
* - consistent up or down regulation in both U87 in vitro and U87 xenografts
↑↓ - opposite response in either U87/HEL or U87-C/U87-X cell types
A-F - cluster of expression in U87 xenografts (see FIG. 1).

TABLE 4

| cDNA | | | 1 hr | 5 hr | 24 hr | 1 hr | 5 hr | 24 hr | 1 hr | 5 hr | 24 hr |
|---|---|---|---|---|---|---|---|---|---|---|---|
| ID | ACC# | Name | 1 gy | 1 gy | 1 gy | 3 gy | 3 gy | 3 gy | 10 gy | 10 gy | 10 gy |
| 1376827 | AA812973 | Testis-specific TCP20 | 3.37 | 5.28 | 6.02 | 6.4 | 10.53 | 11.12 | 15.69 | 16.05 | 16 |
| 385003 | AA709143 | TTF-I | 6.05 | 5.45 | 0.73 | 4.41 | 3.09 | 0.69 | 11.12 | 10.1 | 3.91 |
| 306575 | N94820 | Hepatitis delta antigen | 6.93 | 5.03 | 0.67 | 6.69 | 4.98 | 0.98 | 14.43 | 9.25 | 3.23 |
| | | interacting protein A (dipA) | | | | | | | | | |
| 810444 | AA457114 | B94 protein | 2.82 | 2.19 | 0.39 | 2.04 | 1.53 | 0.27 | 12.41 | 9.02 | 2.07 |
| 27516 | R14080 | Calcium modulating ligand | 4.64 | 3.76 | 1.04 | 5.48 | 3.27 | 1.01 | 11.72 | 9 | 2.78 |
| 124597 | R02373 | Enyol-coA: hydratase | 6.14 | 3.86 | 0.84 | 4.97 | 3.05 | 0.97 | 9.09 | 8.16 | 1.82 |
| | | 3-hydroxyacyl-coA | | | | | | | | | |
| | | dehydrogenase | | | | | | | | | |
| 183476 | H45618 | apM1 GS3109 (novel adipose | 3.52 | 2.12 | 1.49 | 3.62 | 2.99 | 2.12 | 3.05 | 6.67 | 4.46 |
| | | specific collagen-like) | | | | | | | | | |
| 156473 | R73525 | Epoxide hydrolase 2, | 4.34 | 3.9 | 0.31 | 7.48 | 5.46 | 0.36 | 9.26 | 6.48 | 1.64 |
| | | cytoplasmic | | | | | | | | | |
| 741815 | AA402960 | HLA class III region | 9.85 | 10.45 | 28.49 | 3.55 | 7.62 | 19.73 | 6.64 | 6.39 | 26.69 |
| 1412344 | AA844930 | Pancreatic zymogen granule | 6.54 | 4.84 | 1.4 | 4.54 | 2.67 | 1.04 | 7.71 | 5.94 | 1.87 |
| | | membrane protein GP | | | | | | | | | |
| 130541 | R22412 | Platelet/endothelial cell | 7.66 | 7.16 | 8.66 | 3.42 | 2.38 | 3.57 | 6.7 | 5.67 | 7.42 |
| | | adhesion molecule (CD31 a) | | | | | | | | | |
| 124261 | R02346 | U1snRNP 70 K protein | 4.23 | 3.45 | 1.05 | 5.17 | 3.26 | 1.29 | 8.39 | 5.64 | 1.66 |
| 859807 | AA668527 | Mucosal addressin cell | 4.76 | 3.57 | 0.92 | 6.19 | 3.68 | 1.03 | 7.45 | 5.59 | 1.82 |
| | | adhesion molecule-1 (MAd) | | | | | | | | | |
| 511909 | AA088861 | L1-cadherin | 5.17 | 3.9 | 0.99 | 6.63 | 3.96 | 1.65 | 6.12 | 5.57 | 1.45 |
| 246765 | N53169 | Apolipoprotein C-III | 8.49 | 6.72 | 1.79 | 11.28 | 5.89 | 2.04 | 6.49 | 5.42 | 1.24 |
| 502582 | AA134555 | GT198, ORF | 6.06 | 7.01 | 10.69 | 2.15 | 3.4 | 5.27 | 5.39 | 5.3 | 9.96 |
| 138936 | R62817 | Erythrocyte band 7 integral | 4.04 | 3.82 | 1.16 | 4.59 | 2.48 | 0.83 | 6.98 | 5.25 | 2.53 |
| | | membrane protein | | | | | | | | | |
| 272690 | N36174 | 5-hydroxytryptamine 2B | 5.88 | 6.48 | 26.6 | 3.02 | 6.12 | 20.12 | 3.64 | 4.68 | 21.42 |
| | | receptor | | | | | | | | | |
| 306444 | N92711 | Transcription factor TFIID | 4.89 | 3.69 | 0.93 | 4.37 | 3 | 1.02 | 6.21 | 4.35 | 1.31 |
| | | subunit TAFII28 | | | | | | | | | |
| 586854 | AA130874 | Tyrosine phosphatase | 3.52 | 2.85 | 0.28 | 4.99 | 3.05 | 0.91 | 6.31 | 4.82 | 1.23 |
| 84713 | T74257 | Fibrinogen Beta Chain | 3.47 | 2.43 | 0.74 | 1.38 | 1.72 | 0.46 | 4.78 | 4.52 | 1.34 |
| | | Precursor | | | | | | | | | |
| 24415 | R39356 | Tumor protein p53 (Li- | 5.06 | 4.64 | 0.59 | 4.55 | 3 | 0.32 | 6.06 | 4.47 | 0.72 |
| | | Fraumeni syndrome) | | | | | | | | | |
| 382457 | AA069770 | Potassium channel Kv2.1 | 4.51 | 6.36 | 32.59 | 3.07 | 4.73 | 19.67 | 3.32 | 4.43 | 26.73 |
| 858153 | AA633811 | E4BP4 gene | 6.55 | 6.86 | 13.07 | 2.3 | 1.35 | 7.14 | 3.86 | 4.37 | 10.32 |
| 276237 | R94175 | p190-B (p190-B) | 9.32 | 10.33 | 2.29 | 9.11 | 6 | 2.39 | 5.48 | 4.28 | 1.46 |
| 241474 | H90415 | Breast cancer 1, early onset | 5.34 | 4.34 | 1.21 | 3.3 | 2.45 | 0.94 | 5.72 | 4.28 | 1.71 |
| 782488 | AA448468 | MACH-alpha-2 protein | 3.21 | 3.06 | 0.66 | 2.82 | 1.87 | 0.62 | 5.4 | 4.19 | 1.25 |
| 1343971 | AA732873 | Serine/threonine protein | 4.23 | 3.62 | 13.91 | 4.44 | 2.75 | 7.17 | 4.6 | 3.96 | 19.52 |
| | | kinase SAK | | | | | | | | | |
| 108667 | T72628 | Splicing factor SF3a120 | 2.51 | 4.34 | 16.27 | 2.13 | 4.36 | 11.23 | 2.2 | 3.89 | 19.83 |
| 265645 | N31452 | Histamine N-methyltransferase | 3.68 | 3.55 | 1.45 | 6.97 | 4.52 | 2.07 | 5 | 3.82 | 2.03 |
| 840474 | AA485871 | Myosin-I beta | 3.82 | 2.99 | 0.42 | 4.05 | 2.9 | 0.31 | 5.03 | 3.74 | 0.82 |
| 825013 | AA489201 | PHAP12b protein | 10.77 | 9.4 | 2.04 | 5.95 | 3.87 | 1.15 | 6.47 | 3.71 | 1.34 |
| 154015 | R48796 | Integrin, alpha L | 5.22 | 4.33 | 1.28 | 4.7 | 2.46 | 0.88 | 5.12 | 3.66 | 1.13 |
| 1472336 | AA873499 | Class I histocompatibility | 6.48 | 7.46 | 23.87 | 2.82 | 6.02 | 16.11 | 4.1 | 3.62 | 17.25 |
| | | antigen-like protein | | | | | | | | | |
| 884500 | AA629987 | 40 kDa Peptidyl-prolyl | 6.14 | 4.77 | 0.27 | 4.83 | 2.63 | 0.12 | 5.16 | 3.59 | 0.1 |
| | | cis-trans Isomerase | | | | | | | | | |
| 232772 | H72723 | Metallothionein I-B gene | 1.21 | 1.39 | 0.33 | 3.91 | 1.58 | 0.79 | 4.45 | 3.59 | 0.72 |
| 562115 | AA211508 | Zinc finger protein 139 | 5.29 | 4.01 | 0.92 | 4.42 | 3.44 | 0.85 | 3.84 | 3.55 | 1 |
| | | (clone pHZ-37) | | | | | | | | | |
| 725076 | AA404619 | 5′ nucleotidase (CD73) | 3.02 | 2.53 | 0.04 | 4.3 | 3.04 | 0.71 | 3.7 | 3.55 | 0.09 |
| 204686 | H57136 | Phospholemman chloride | 2.02 | 1.45 | 6.64 | 1.98 | 2.6 | 18.78 | 3.85 | 3.47 | 20.8 |
| | | channel | | | | | | | | | |
| 67769 | T49657 | TWIK-related acid-sensitive | 4.84 | 3.19 | 1.34 | 3.65 | 2.45 | 1.1 | 3.89 | 3.46 | 1.4 |
| | | K+ channel (TASK) | | | | | | | | | |
| 1032431 | AA779480 | Bone morphogenetic protein 8 | 2.45 | 2.02 | 0.72 | 3.37 | 2.81 | 1.11 | 3.63 | 3.45 | 1.06 |
| | | (osteogenic protein 2) | | | | | | | | | |
| 742115 | AA405800 | Dodecenoyl-Coenzyme A delta | 5.11 | 5.36 | 13.77 | 3 | 5.18 | 13.45 | 3.51 | 3.44 | 15.56 |
| | | isomerase | | | | | | | | | |
| 51814 | H22919 | Cystatin B | 8.66 | 7.11 | 3.48 | 5.55 | 2.9 | 1.77 | 4.23 | 3.33 | 1.55 |
| 768260 | AA424950 | Retinoblastoma Binding | 4.59 | 5.04 | 6.99 | 2.56 | 3.4 | 4.7 | 3.03 | 3.27 | 6.04 |
| | | Protein 1 | | | | | | | | | |
| 811813 | AA443039 | Heat shock 70 kD protein 1 | 4.59 | 5.04 | 6.99 | 2.56 | 3.4 | 4.7 | 3.13 | 3.27 | 6.04 |
| 461516 | AA705069 | Receptor of retinoic acid | 1.04 | 1.17 | 4.82 | 1.32 | 1.34 | 6.05 | 3.17 | 3.27 | 19.29 |
| 415529 | W80632 | BRCA2 region, sequence CG006 | 3.45 | 2.41 | 0.77 | 4.62 | 3.63 | 0.25 | 4.35 | 3.26 | 0.89 |
| 287687 | N59150 | Interferon-alpha/beta | 3.4 | 3.08 | 0.64 | 4.66 | 2.8 | 0.43 | 3.03 | 3.17 | 0.71 |
| | | receptor alpha | | | | | | | | | |
| 324861 | W48713 | Epidermal growth factor | 17.69 | 16.35 | 25.92 | 2.18 | 3.41 | 4.25 | 3.14 | 3.15 | 6.18 |
| | | receptor | | | | | | | | | |
| 135085 | R33031 | Sigma 3B protein | 4.02 | 3.16 | 0.82 | 5.31 | 3.14 | 1.11 | 3.71 | 3.13 | 1 |
| 825323 | AA504477 | Cytoskeleton associated | 4.25 | 5.04 | 6.87 | 2.23 | 3.71 | 4.51 | 3.4 | 3.08 | 6.26 |
| | | protein (CG22) | | | | | | | | | |
| 841221 | AA486741 | Argininosuccinate lyase | 4.2 | 3.97 | 1 | 4.53 | 3.41 | 1.43 | 3.75 | 3.02 | 1.12 |
| 148444 | H12320 | cAMP-response element | 3.12 | 2.78 | 0.96 | 2.84 | 1.81 | 1.02 | 3.98 | 2.96 | 1.21 |
| | | binding protein | | | | | | | | | |
| 838389 | AA458807 | Retinal protein (HRG4) | 4.63 | 5.07 | 8.75 | 2.63 | 3.26 | 5.59 | 3.33 | 2.95 | 6.07 |
| 236059 | H53703 | Squamous cell carcinoma of | 3.68 | 4.38 | 7.61 | 2.52 | 4.04 | 5.98 | 2.56 | 2.93 | 6.6 |
| | | esophagus GRB-7 SH | | | | | | | | | |
| 610097 | AA171449 | Biphenyl hydrolase-related | 2.51 | 2.57 | 0.81 | 4.77 | 3.08 | 1.11 | 3.77 | 2.92 | 1 |
| | | protein | | | | | | | | | |
| 590759 | AA157955 | Methyl sterol oxidase (ERG25) | 4.44 | 3.12 | 0.9 | 4.4 | 2.64 | 0.95 | 3.58 | 2.92 | 0.76 |
| 815303 | AA481562 | Aspartyl-t synthetase | 2.72 | 2.82 | 0.64 | 2.13 | 1.72 | 0.31 | 3.73 | 2.9 | 1.3 |
| 146868 | R80779 | Protein kinase (MLK-3) | 6.25 | 5.82 | 11.6 | 2.16 | 3.94 | 5.76 | 2.71 | 2.89 | 6.67 |
| 41607 | R54176 | Von Hippel-Lindau syndrome | 4.78 | 3.73 | 0.95 | 3.85 | 2.38 | 0.91 | 4.2 | 2.89 | 1.08 |
| 190491 | H37774 | Tuberin | 3.38 | 2.36 | 0.52 | 3.69 | 2.36 | 1.09 | 3.19 | 2.87 | 0.96 |
| 897594 | AA496879 | (clone E5.1) RNA-binding | 5.42 | 5.19 | 8.32 | 2.33 | 4.7 | 6.29 | 2.98 | 2.85 | 6.18 |
| | | protein | | | | | | | | | |
| 769948 | AA430512 | Cytoplasmic antiproteinase | 2.56 | 3.39 | 3.84 | 2.69 | 3.28 | 4.05 | 2.44 | 2.81 | 4.15 |
| | | 3 (CAP3) | | | | | | | | | |
| 758366 | AA404293 | Triadin | 6.18 | 7.38 | 11.97 | 1.97 | 3.41 | 4.66 | 2.47 | 2.8 | 6.53 |
| 843139 | AA485922 | Copine I | 2.39 | 2.13 | 0.67 | 2.85 | 1.95 | 0.69 | 3.93 | 2.79 | 0.91 |
| 771295 | AA443634 | Ubiquitin conjugating | 2.89 | 2.35 | 0.66 | 7.08 | 4.54 | 1.49 | 3.4 | 2.77 | 1.03 |
| | | enzyme G2 (UBE2G2) | | | | | | | | | |
| 323500 | W45688 | Cysteine protease Mch2 | 2.62 | 2.13 | 0.67 | 3.48 | 1.86 | 0.69 | 3.03 | 2.75 | 0.98 |
| | | isoform alpha (Mch2) | | | | | | | | | |
| 814378 | AA458849 | Placental bikunin | 6 | 7.16 | 13.35 | 1.66 | 2.71 | 5.21 | 2.39 | 2.74 | 7.45 |
| 586706 | AA160584 | Carcinoembryonic antigen | 1.99 | 4.72 | 0.06 | 24.10 | 16.94 | 6.08 | 2.68 | 2.72 | 1.22 |
| | | precursor | | | | | | | | | |
| 267865 | N34095 | FEZ2 | 2.69 | 1.76 | 0.69 | 3.18 | 1.73 | 0.97 | 2.78 | 2.7 | 1.35 |
| 838359 | AA458785 | Guanylate cyclase soluble | 2.85 | 2.14 | 0.61 | 2.92 | 1.8 | 0.71 | 3.75 | 2.69 | 0.8 |
| | | beta-1 chain | | | | | | | | | |
| 174627 | H27864 | Secretogranin II precursor | 1.83 | 2.22 | 18.71 | 1.27 | 3.37 | 20.47 | 2.67 | 2.68 | 25.35 |
| 144932 | R78607 | Putative oral tumor | 4.01 | 4.62 | 12.79 | 2.31 | 3.26 | 7.89 | 3.06 | 2.68 | 10.11 |
| | | suppressor protein (doc-1) | | | | | | | | | |
| 302310 | N78843 | Cyclophilin-33A (CYP-35) | 3.36 | 4.58 | 11.18 | 1.91 | 3.1 | 5.26 | 2.5 | 2.56 | 5.77 |
| 35318 | R45428 | DNAJ protein homolog 2 | 2.49 | 2.34 | 0 | 3.08 | 1.7 | 0.22 | 4.14 | 2.65 | 1.3 |
| 841149 | AA437034 | Transforming growth factor, | 3.43 | 4.06 | 7.98 | 2.06 | 3 | 4.36 | 2.93 | 2.68 | 7.92 |
| | | beta receptor H | | | | | | | | | |
| | | 70-80 k | | | | | | | | | |
| 796984 | AA463492 | Chronic granulomatous | 4.56 | 4.77 | 14.44 | 2.48 | 5.2 | 14.940 | 2.81 | 2.58 | 11.43 |
| | | disease | | | | | | | | | |
| 771303 | AA443638 | Breast cancer-specific | 2.72 | 3.31 | 11.99 | 2.42 | 3.53 | 13.13 | 2.8 | 2.57 | 12 |
| | | protein 1 (BCSG1) | | | | | | | | | |
| 810040 | AA455272 | ITAB1 protein | 4.34 | 5.13 | 16.27 | 2.92 | 4.27 | 12.81 | 2.82 | 2.56 | 11.44 |
| 487373 | AA046701 | ATP synthase lipid-binding | 3.77 | 4.74 | 13 | 3.24 | 4.12 | 10.52 | 2.67 | 2.55 | 11.73 |
| | | protein P1 PR1 | | | | | | | | | |
| 324891 | W49667 | Putative fatty acid | 2.75 | 2.46 | 1.33 | 3.38 | 2.49 | 1.62 | 3.16 | 2.49 | 1.81 |
| | | desaturase MLD | | | | | | | | | |
| 842860 | AA486393 | Cytokine receptor family | 3.96 | 3.25 | 1.25 | 3.41 | 2.41 | 1.11 | 3.09 | 2.48 | 0.97 |
| | | II, member 4 | | | | | | | | | |
| 347434 | W81191 | Nucleolar autoantigen | 3.48 | 2.99 | 0.83 | 3.14 | 1.87 | 0.7 | 3.29 | 2.48 | 1.04 |
| | | No 55 | | | | | | | | | |
| 755750 | AA496628 | Non-metastatic cells 2, | 0.87 | 1.11 | 0.29 | 1.07 | 0.83 | 0.6 | 2.39 | 2.47 | 1.38 |
| | | protein (NM23B) expressed | | | | | | | | | |
| 840364 | AA485626 | S-adenosylhomocysteine | 3.53 | 3.3 | 5.03 | 1.7 | 3.12 | 3.82 | 2.87 | 2.46 | 4.89 |
| | | hydrolase | | | | | | | | | |
| 305606 | N90246 | Tyrosine-protein kinase | 4.33 | 4.79 | 10.92 | 1.64 | 2.69 | 5.56 | 1.95 | 2.46 | 6.12 |
| | | receptor EPH P | | | | | | | | | |
| 840753 | AA486072 | Small inducible cytokinase | 2.66 | 2.63 | 0.99 | 2.73 | 1.68 | 1 | 3.08 | 2.44 | 1.11 |
| | | A5 (RANTES) | | | | | | | | | |
| 725473 | AA397819 | NKG2-D type II integral | 7.7 | 8.95 | 16.63 | 1.98 | 3.75 | 5.12 | 2.66 | 2.43 | 6.08 |
| | | membrane protein | | | | | | | | | |
| 382693 | AA069414 | Glial fibrillary acidic | 4.89 | 4.97 | 10.8 | 2.07 | 4.55 | 6.75 | 2.12 | 2.42 | 6.7 |
| | | protein | | | | | | | | | |
| 1461664 | AA885311 | Butyrylcholinesterase | 4.26 | 4.72 | 6.92 | 1.56 | 2.56 | 3.72 | 2.59 | 2.41 | 4.6 |
| 146577 | R79935 | TGF-beta inducible early | 5.53 | 7.81 | 27.64 | 3.19 | 5.75 | 18.44 | 1.67 | 2.39 | 11.54 |
| | | protein (TIEG) | | | | | | | | | |
| 69184 | T54144 | Homolog of the Aspergillus | 4.51 | 3.36 | 0.81 | 3.8 | 2.36 | 0.77 | 3.18 | 2.39 | 0.97 |
| | | nidulans sudD gene product | | | | | | | | | |
| 854668 | AA630082 | Cyclin-dependent kinase | 3.12 | 2.36 | 0.06 | 5.35 | 2.93 | 0.41 | 3.18 | 2.38 | 0.34 |
| | | inhibitor p27kip1 | | | | | | | | | |
| 78353 | T56281 | Metallothionein (MT)I-F | 2.23 | 1.42 | 0.12 | 4.35 | 1.74 | 0.88 | 2.17 | 2.38 | 0.76 |
| | | gene | | | | | | | | | |
| 745347 | AA625666 | Pig7 (PIG7) | 3.49 | 3.86 | 4.26 | 1.67 | 2.79 | 2.74 | 2.24 | 2.34 | 3.87 |
| 272327 | N32199 | Melanoma antigen recognized | 3.22 | 2.5 | 3.02 | 3.81 | 2.68 | 4.46 | 2.93 | 2.34 | 3.3 |
| | | by T-cells (MART-1) | | | | | | | | | |
| 123980 | R01638 | HYA22 | 4.7 | 4.87 | 7.22 | 1.96 | 2.67 | 3.18 | 2.15 | 2.34 | 4.3 |
| 743229 | AA400329 | Gene neurofilament subunit | 4.16 | 4.84 | 35.8 | 3.29 | 6.1 | 42.03 | 2.08 | 2.32 | 23.26 |
| | | M (NF-M) | | | | | | | | | |
| 81417 | T60163 | Ribonuclease L (2′,5′- | 5.1 | 3.71 | 6.01 | 3.95 | 6.05 | 8.46 | 2.32 | 2.31 | 4.22 |
| | | oligoisoadenylate synthetase) | | | | | | | | | |
| 341978 | W61361 | Cytoplasmic antiproteinase | 3.72 | 3.53 | 11.83 | 1.68 | 4.83 | 14.04 | 2.73 | 2.31 | 11.22 |
| | | 2 (CAP2) | | | | | | | | | |
| 45645 | H08753 | G protein beta 5 subunit | 4.39 | 3.05 | 0.85 | 5.08 | 2.74 | 1.07 | 3.21 | 2.29 | 0.87 |
| 415145 | W95082 | Hydroxysteroid (11-beta) | 8.55 | 8.62 | 11.6 | 3.16 | 3.72 | 5.28 | 2.91 | 2.28 | 3.91 |
| | | dehydrogenase 2 | | | | | | | | | |
| 759873 | AA423870 | p37NB | 4.17 | 3.88 | 8.52 | 2.47 | 4.12 | 7.92 | 2.31 | 2.27 | 5.3 |
| 302632 | N90281 | B7 | 7.34 | 8.33 | 27.23 | 3.93 | 7.55 | 20.05 | 1.99 | 2.26 | 13.4 |
| 773344 | AA425395 | X-linked PEST-containing | 5.41 | 6.15 | 16.47 | 1.56 | 3.6 | 8.82 | 1.87 | 2.23 | 9.28 |
| | | transporter (XPCT) | | | | | | | | | |
| 297895 | N70057 | LST1, cLST1/A splice variant | 4.72 | 7.1 | 31.27 | 1.66 | 2.84 | 11.13 | 3.08 | 2.23 | 14.98 |
| 268876 | N26026 | Survival of motor neuron | 2.92 | 2.72 | 0.99 | 3.2 | 2.18 | 0.98 | 2.77 | 2.21 | 0.92 |
| | | protein interacting | | | | | | | | | |
| | | protein | | | | | | | | | |
| 345559 | W73892 | Putative tumor suppressor | 5.19 | 5.62 | 16.67 | 2.14 | 3.81 | 10.78 | 2.4 | 2.18 | 10.09 |
| | | (LUCA15) | | | | | | | | | |
| 859422 | AA666180 | v-erbA related ear-2 gene | 3 | 2.61 | 1.01 | 2.26 | 1.64 | 0.91 | 2.72 | 2.17 | 1.14 |
| 1416782 | AA894557 | Creatine kinase B | 4.05 | 4.3 | 7.44 | 1.73 | 2.8 | 3.79 | 2.19 | 2.14 | 4.52 |
| 1412238 | AA844818 | Amylase, alpha 2A; pancreatic | 2.57 | 2.82 | 16.75 | 1.64 | 3.57 | 15.14 | 2.52 | 2.11 | 16.7 |
| 755037 | AA411324 | IL-13Ra | 2.13 | 1.89 | 21.84 | 1.49 | 1.96 | 13.81 | 2.39 | 2.1 | 17.67 |
| 773203 | AA428551 | SOX22 protein (SOX22) | 3.39 | 3.14 | 5.79 | 1.44 | 2.28 | 3.35 | 1.59 | 2.07 | 3.81 |
| 704760 | AA282537 | Myocyte-specific enhancer | 3.88 | 5.14 | 7.29 | 1.71 | 2.94 | 3.85 | 2.1 | 2.06 | 3.37 |
| | | factor 2 | | | | | | | | | |
| 713886 | AA284856 | Adult heart neutral calpenin | 2.95 | 2.63 | 3.12 | 2.46 | 2.38 | 3.21 | 2.32 | 2.06 | 3.02 |
| 562883 | AA0886619 | RLIP76 protein | 1.19 | 1.52 | 12.34 | 1.15 | 1.92 | 11.13 | 1.35 | 2.06 | 12.86 |
| 744940 | AA625888 | Acrosin-trypsin inhibitor II | 4.27 | 5.2 | 26.78 | 1.73 | 5.22 | 20.38 | 1.3 | 1.96 | 14.69 |
| | | precursor | | | | | | | | | |
| 783836 | AA43659 | Zinc finger protein 143 | 4.72 | 4.47 | 7.73 | 1.89 | 2.61 | 1.4 | 2.17 | 1.96 | 4.56 |
| | | (clone pHZ-1) | | | | | | | | | |
| 769600 | AA425900 | Uracil-DNA glycosylase | 3.91 | 4.0 | 6.92 | 1.6 | 2.44 | 3.15 | 1.85 | 1.95 | 3.57 |
| 279970 | N57553 | Adenosine receptor A2 | 6.77 | 7.79 | 16.45 | 1.44 | 2.66 | 6.87 | 2.04 | 1.93 | 5.91 |
| 774754 | AA442092 | Catenin (cadherin-associated | 4.4 | 5.08 | 12.99 | 1.76 | 3.23 | 7.88 | 1.77 | 1.91 | 4.81 |
| | | protein), beta 1 (88 kDa) | | | | | | | | | |
| 810725 | AA457717 | Proton-ATPase-like protein | 4.66 | 4.96 | 10.11 | 1.45 | 2.19 | 5.55 | 2.02 | 1.91 | 5.92 |
| 884867 | AA669443 | Eukaryotic translation | 2.12 | 2.45 | 6.32 | 1.1 | 1 | 2.99 | 1.34 | 1.89 | 2.94 |
| | | initiation factor 5 (eIF5) | | | | | | | | | |
| 298062 | N70734 | Troponin T2 (cardiac) | 3.89 | 4.35 | 6.84 | 1.47 | 2.02 | 2.82 | 1.7 | 1.87 | 3.77 |
| 273546 | N33274 | Multifunctional protein ADE2 | 2.7 | 3.32 | 13.19 | 1.5 | 2.37 | 10.33 | 2.12 | 1.85 | 14.09 |
| 259579 | N29765 | RAD51D | 4.55 | 4.68 | 7 | 1.56 | 2.49 | 3.39 | 1.71 | 1.84 | 3.31 |
| 814316 | Aa459104 | 60 Ribosomal protein L13 | 2.8 | 2.69 | 6.51 | 1.61 | 2.3 | 4.95 | 1.84 | 1.83 | 4.09 |
| 1476065 | Aa873060 | Stathmin | 3.22 | 3.32 | 4.26 | 1.5 | 2.1 | 2.51 | 1.49 | 1.83 | 3.17 |
| 148231 | H13691 | Major histocompatibility | 15.2 | 16.77 | 93.91 | 1.65 | 2.73 | 14.49 | 1.73 | 1.79 | 12.37 |
| | | complex, class II, DM beta | | | | | | | | | |
| 740914 | AA478268 | CtBP | 1.41 | 2.09 | 2.75 | 2.22 | 3.19 | 3.65 | 1.65 | 1.78 | 2.94 |
| 810801 | AA458878 | Agrin precursor | 1.2 | 0.75 | 10.77 | 1.48 | 2.41 | 12.6 | 0.47 | 1.78 | 12.98 |
| 429182 | AA004759 | Dolichol monophosphate | 4.85 | 5.14 | 9.46 | 1.24 | 2.64 | 4.29 | 1.73 | 1.77 | 4.39 |
| | | mannose synthase (DPM1) | | | | | | | | | |
| 950709 | AA608583 | OTK27 | 2.72 | 2.82 | 3.2 | 2.14 | 2.97 | 2.84 | 1.73 | 1.76 | 2.7 |
| 252259 | H87536 | Bullous pemphigoid antigen 2 | 2.57 | 3.19 | 3.43 | 1.53 | 2.07 | 2.36 | 1.81 | 1.76 | 2.36 |
| | | (180 kDa) | | | | | | | | | |
| 824426 | AA490300 | PDGF associated protein | 2.31 | 2.61 | 2.81 | 1.63 | 1.91 | 2.09 | 1.8 | 1.76 | 2.39 |
| 122159 | T98612 | Alpha-1 type 3 collagen | 1.06 | 0.72 | 0.28 | 2.17 | 1.28 | 0.39 | 1.89 | 1.75 | 0.28 |
| 39145 | R51835 | Unknown EST | 3.53 | 2.78 | 0.24 | 8.25 | 4.39 | 1.04 | 3.03 | 1.72 | 0.66 |
| 756931 | AA425934 | S100 alpha protein | 3.43 | 3.19 | 4.94 | 1.24 | 1.6 | 2.17 | 1.42 | 1.71 | 2.88 |
| 1412502 | AA845167 | Elastase IIIA precursor | 2.12 | 2.86 | 28.34 | 0.97 | 1.46 | 9.49 | 2.3 | 1.68 | 16.04 |
| 588559 | AA147043 | CAGH1a (CAGH1) | 3.39 | 3.56 | 9.01 | 2.12 | 2.7 | 6.43 | 1.61 | 1.64 | 5.61 |
| 297392 | N80129 | Metallothionein 1L | 1.95 | 1.23 | 0.38 | 3.74 | 1.42 | 0.76 | 2.13 | 1.62 | 0.25 |
| 712577 | AA281549 | Putative holocytochrome | 2.47 | 2.9 | 2.41 | 1.5 | 2.38 | 1.78 | 1.55 | 1.58 | 1.82 |
| | | c-type synthetase | | | | | | | | | |
| 866882 | AA679352 | FarnesyL-Diphosphate | 2.05 | 2.26 | 5.53 | 1.27 | 1.79 | 4.07 | 2.28 | 1.58 | 6.03 |
| | | Farnesyltransferase | | | | | | | | | |
| 687054 | AA258001 | Transcription factor RELb | 4.23 | 4.93 | 29.73 | 2.41 | 4.62 | 23.12 | 1.47 | 1.57 | 15.39 |
| 744917 | AA625806 | Ninjurin 1 | 2.73 | 2.82 | 17.96 | 2.5 | 4.78 | 27.93 | 1.21 | 1.54 | 13.76 |
| 884790 | AA629838 | Zinc finger protein 74 (Cos52) | 2.58 | 2.67 | 3.23 | 1.27 | 1.64 | 1.8 | 1.7 | 1.54 | 2 |
| 771220 | AA443547 | Transcription factor P65 | 2.61 | 2.28 | 22.94 | 2.14 | 3.49 | 25.52 | 1.22 | 1.52 | 14.69 |
| 1470333 | AA866113 | Fe65-like protein (hFE65L) | 2.61 | 3.1 | 4.02 | 1.28 | 1.96 | 2.27 | 1.5 | 1.52 | 2.79 |
| 809578 | AA456616 | Ribosomal protein S5 | 2.6 | 2.48 | 3.96 | 1.76 | 2.59 | 3.22 | 1.29 | 1.5 | 2.86 |
| 39993 | R52548 | Superoxide dismutase (SOD-1) | 2.505 | 3.025 | 4.66 | 1.245 | 1.94 | 2.755 | 1.33 | 1.485 | 2.97 |
| 310138 | N98485 | Khead protein FREAC-2 | 2.42 | 1.78 | 7.69 | 1.25 | 2.15 | 5.7 | 1.15 | 1.43 | 5.08 |
| 813751 | AA453813 | Gal-beta (1-3/1-4) GlcNAc | 1.58 | 1.72 | 11.32 | 3.45 | 4.07 | 28.05 | 1.15 | 1.41 | 12.14 |
| | | alpha-2,3sialytransferase | | | | | | | | | |
| 782427 | AA431832 | Granulin | 2.9 | 3.47 | 4.49 | 1.5 | 2.31 | 3.18 | 1.56 | 1.41 | 2.74 |
| 825451 | AA504342 | p115 | 2.71 | 3 | 3.59 | 1.26 | 1.9 | 2.24 | 1.22 | 1.4 | 2.24 |
| 711826 | AA281057 | Ribosomal protein S17 | 4.1 | 5.47 | 7.65 | 1.74 | 3.08 | 3.95 | 1.58 | 1.39 | 2.86 |
| 771084 | AA427906 | GT197 partial ORF, 3 end of cds | 2.68 | 2.85 | 15.02 | 1.19 | 2.57 | 11.3 | 1.85 | 1.36 | 8.37 |
| 208161 | H62527 | Tyrosine 3-monooxygenase | 3.15 | 2.42 | 0.7 | 2.82 | 1.71 | 0.73 | 1.82 | 1.36 | 0.57 |
| | | activation protein | | | | | | | | | |
| 361974 | AA001449 | Pleiotrophin | 0.77 | 1.01 | 0.25 | 4.49 | 1.28 | 0.57 | 1.47 | 1.36 | 0.21 |
| 788421 | AA456439 | Homozygous deletion target in | 2.27 | 2.95 | 3.55 | 1 | 1.66 | 1.87 | 1.19 | 1.33 | 2.28 |
| | | pancreatic carcinoma | | | | | | | | | |
| 415817 | W84868 | Cytochrome P450, subfamily | 2.52 | 3.02 | 4.37 | 4.36 | 2.06 | 3.47 | 1.44 | 1.3 | 2.94 |
| | | IVA, polypeptide 11 | | | | | | | | | |
| 278650 | N66208 | (ard-1) | 2 | 2.18 | 2.63 | 1.03 | 1.36 | 1.71 | 1.54 | 1.29 | 1.84 |
| 711474 | AA401111 | Glucose phosphate isomerase | 2.45 | 3.43 | 4.19 | 1.32 | 1.86 | 2.24 | 1.52 | 1.28 | 2.26 |
| 757440 | AA437226 | Interleukin 10 receptor | 2.59 | 2.7 | 3.54 | 1.15 | 1.59 | 2.31 | 1.25 | 1.26 | 1.84 |
| 302933 | N90109 | Alpha-cardiac actin gene, | 4.33 | 1.02 | 8.5 | 7.05 | 6.44 | 13.62 | 1.88 | 1.23 | 3.21 |
| | | 5′ flank | | | | | | | | | |
| 23185 | R39239 | Hexabrachion (tenascin C, | 0.78 | 0.74 | 0.24 | 1.1 | 1.12 | 0.37 | 1.18 | 1.21 | 0.37 |
| | | cytotactin) | | | | | | | | | |
| 1472643 | AA872341 | 40S Ribosomal protein S15A | 1.93 | 1.55 | 0.23 | 2.02 | 0.98 | 0.29 | 1.78 | 1.2 | 0.26 |
| 839890 | AA490047 | Alpha-SPI | 01 | 0.86 | 0.27 | 3.78 | 1.54 | 0.4 | 1.91 | 1.18 | 0.44 |
| 327350 | W02101 | Heterogeneous nuclear | 1.53 | 1.54 | 0.37 | 1.51 | 0.7 | 0.32 | 2.05 | 1.17 | 0.6 |
| | | ribonucleoprotein A2/B1 | | | | | | | | | |
| 756600 | AA481464 | Peptidylprolyl isomerase B | 1.29 | 1.55 | 5.05 | 1.86 | 1.86 | 7.36 | 1.51 | 1.15 | 4.89 |
| | | (cyclophilin B) | | | | | | | | | |
| 345063 | W72250 | Calcium channel beta-1 B1 | 0.37 | 0 | 0.1 | 0.5 | 1.19 | 1.4 | 1.52 | 1.15 | 0.46 |
| | | subunit | | | | | | | | | |
| 884822 | AA669341 | (p23) | 0.97 | 0.82 | 0.39 | 1.68 | 0.69 | 0.23 | 2.81 | 1.15 | 0.4 |
| 344039 | W70051 | M-phase phosphoprotein, mpp9 | 3.11 | 2.73 | 25.07 | 1.7 | 2.77 | 17.66 | 0.83 | 1.14 | 8.51 |
| 470175 | AA029308 | MTCP1 gene, exons 2A to 7 | 2.59 | 2.79 | 4.39 | 1.28 | 1.96 | 2.97 | 1.21 | 1.14 | 1.85 |
| | | (and joined) | | | | | | | | | |
| 665373 | AA195036 | Ro/SSA ribonucleoprotein | 1.02 | 0.91 | 14.44 | 1.36 | 1.88 | 34.85 | 0.57 | 1.13 | 13.56 |
| | | homolog (RoRet) | | | | | | | | | |
| 269606 | N26769 | N-methylpurine-DNA glycosylase | 2.75 | 3.15 | 3.67 | 1.19 | 1.8 | 2.09 | 0.87 | 1.13 | 1.78 |
| 509887 | AA056465 | 54 kDa protein | 1.16 | 0.83 | 3.87 | 1.38 | 0.98 | 4.11 | 0.82 | 1.13 | 1.98 |
| 768299 | AA424743 | ERF-1 3′ end | 0.93 | 1.05 | 0.34 | 1.91 | 0.96 | 0.27 | 1.09 | 1.11 | 0.36 |
| 1375309 | AA815407 | Ryanodine receptor, skeletal | 0.83 | 0.84 | 3.37 | 1.25 | 1.56 | 6.38 | 1.93 | 1.09 | 3.47 |
| | | muscle | | | | | | | | | |
| 298965 | N71160 | Cytochrome C oxidase | 1.46 | 2.66 | 1.12 | 3.75 | 1.23 | 0.27 | 0.63 | 1.09 | 0 |
| | | subunit Vlb | | | | | | | | | |
| 669419 | AA253413 | Friedreich ataxia | 0.56 | 0 | 1.18 | 0 | 0.84 | 3.23 | 0.32 | 1.08 | 1.79 |
| 53316 | R15814 | Malate dehydrogenase (MDHA) | 0.96 | 1.14 | 0.41 | 1.42 | 0.57 | 0.32 | 1.31 | 1.06 | 0.55 |
| 839991 | AA490172 | Collagen, type I, alpha-2 | 0.72 | 0.29 | 0 | 2.18 | 0.35 | 0 | 1.62 | 1.06 | 0 |
| 810142 | AA464246 | Major histocompatibility | 1.11 | 0.95 | 0.32 | 1.6 | 0.95 | 0.37 | 1.35 | 1.04 | 0.33 |
| | | complex, class I, C | | | | | | | | | |
| 897690 | AA598758 | Homologue of mouse tumor | 0.88 | 0.56 | 0.14 | 1.37 | 0.68 | 0.07 | 1.86 | 1.04 | 0.32 |
| | | rejection antigen gp96 | | | | | | | | | |
| 854879 | AA630354 | Albumin D-box binding protein | 1.2 | 2.09 | 11.46 | 1.28 | 2.8 | 17.27 | 1.55 | 1.03 | 10.15 |
| 868368 | AA634103 | Thymosin beta-4 | 1.34 | 1.12 | 0.3 | 1.7 | 0.88 | 0.17 | 1.52 | 1.03 | 0.24 |
| 43231 | H05893 | 26S proteaseome subunit p97 | 0.78 | 0.83 | 0.18 | 0.75 | 0.41 | 0.13 | 0.94 | 1.02 | 0.35 |
| 1472735 | AA872383 | Metallothionein-le gene | 1.24 | 0.98 | 0.22 | 1.78 | 1.14 | 0.35 | 1.38 | 1.01 | 0.14 |
| | | (hMT-le) | | | | | | | | | |
| 868304 | AA634006 | Actin alpha 2, smooth | 1.33 | 1.09 | 0.31 | 1.75 | 0.9 | 0.38 | 1.44 | 1.01 | 0.35 |
| | | muscle, aorta | | | | | | | | | |
| 377461 | AA055835 | Caveolin, caveolae protein, | 1.01 | 0.84 | 0.26 | 1.43 | 0.72 | 0.3 | 1.15 | 1.01 | 0.52 |
| | | 22kD | | | | | | | | | |
| 490995 | AA136707 | Lysyl hydroxylase insoform | 0.46 | 0.27 | 0 | 1.91 | 1.31 | 0.15 | 1.65 | 0.99 | 0 |
| | | 2 (PLOD2) | | | | | | | | | |
| 950607 | AA608548 | SET protein | 0.95 | 0.72 | 0.09 | 1.11 | 0.83 | 0.15 | 1.3 | 0.99 | 0.31 |
| 781704 | AA431321 | Thyroid receptor interactor | 0.81 | 0.48 | 0.2 | 3.18 | 0.86 | 0 | 0.38 | 0.98 | 0 |
| | | (TRIP7), 3′ end of cds | | | | | | | | | |
| 79502 | T59245 | S-adenosylmethionine | 1.04 | 1.15 | 0.15 | 1.06 | 0.76 | 0.14 | 2.14 | 0.98 | 0.27 |
| | | synthetase gamma | | | | | | | | | |
| 785933 | AA449715 | Sushi-repeat-containing | 0.59 | 0.58 | 0.35 | 2.78 | 1.49 | 0.26 | 1.34 | 0.94 | 0.33 |
| | | protein precursor (SRPX), | | | | | | | | | |
| 392622 | AA708298 | ATP synthase H+ trans- | 0.74 | 0.97 | 0.23 | 1.88 | 0.86 | 0.33 | 1.52 | 0.94 | 0.24 |
| | | porting, beta | | | | | | | | | |
| 971367 | AA683050 | 40S Ribosomal protein S8 | 1.25 | 1.28 | 0.18 | 1.32 | 0.85 | 0.35 | 1.21 | 0.94 | 0.31 |
| 1471829 | AA873351 | Ribosomal protein L35a | 1.34 | 1.05 | 0.38 | 1.25 | 0.81 | 0.54 | 1.32 | 0.93 | 0.26 |
| 80399 | T65786 | Pre-mRNA splicing factor SF2, | 0.43 | 0.8 | 5.45 | 0.99 | 1.09 | 7.07 | 1.12 | 0.92 | 5.73 |
| | | P33 subunit | | | | | | | | | |
| 684661 | AA251930 | Glioma pathogenesis-related | 1.55 | 1.22 | 0.88 | 1.4 | 0.87 | 0.35 | 1.16 | 0.92 | 0.3 |
| | | protein (GliPR) | | | | | | | | | |
| 144881 | R78585 | Calumenin | 0.69 | 0.55 | 0.29 | 1.26 | 0.72 | 0.38 | 1.12 | 0.92 | 0.35 |
| 263716 | H99676 | Collagen, type VI, alpha 1 | 0.86 | 0.73 | 0.25 | 1.88 | 1.01 | 0.57 | 1.02 | 0.91 | 0.23 |
| 810743 | AA457726 | Myelodysplasia/myeloid | 0.55 | 0.63 | 0.18 | 2.49 | 0.95 | 0.39 | 0.78 | 0.91 | 0.28 |
| | | leukemia factor 2 (MLF2) | | | | | | | | | |
| 756687 | AA443899 | Encoding CKA-1 | 2.89 | 2.54 | 44.04 | 0.66 | 1.48 | 12.9 | 0.29 | 0.9 | 12.38 |
| 754538 | AA406285 | Dr1-associated corepressor | 2.27 | 2.21 | 21.33 | 0.92 | 1.18 | 9.51 | 1.22 | 0.89 | 8.56 |
| | | (DRAP1) | | | | | | | | | |
| 34357 | R44290 | Cytoplasmic beta-actin gene | 0.97 | 0.88 | 0.285 | 1.915 | 1.035 | 0.435 | 1.245 | 0.885 | 0.355 |
| 868308 | AA634008 | 40S Ribosomal protein S23 | 1.01 | 0.94 | 0.17 | 2.7 | 1.4 | 0.53 | 1.48 | 0.88 | 0.25 |
| 23019 | R43581 | Guanine nucleotide-binding | 0.69 | 0.8 | 0 | 1.7 | 0.74 | 0.1 | 1.57 | 0.88 | 0.65 |
| | | protein G-s alpha subunit | | | | | | | | | |
| 745138 | AA626698 | Alpha-tubulin isotype | 0.9 | 0.89 | 0.29 | 1.18 | 0.9 | 0.29 | 1.43 | 0.87 | 0.2 |
| | | H2-gene, last exon | | | | | | | | | |
| 855620 | AA664241 | Alpha NAC | 0.76 | 0.64 | 0.15 | 1.64 | 0.36 | 0.05 | 1.21 | 0.86 | 0.03 |
| 786680 | AA451895 | Annexin V (endonexin II) | 0.91 | 0.83 | 0.26 | 1.44 | 0.86 | 0.29 | 1.35 | 0.85 | 0.41 |
| 34396 | R44334 | 90 kDa heat shock protein | 1.02 | 0.87 | 0.275 | 1.62 | 0.855 | 0.28 | 1.245 | 0.84 | 0.245 |
| | | gene | | | | | | | | | |
| 39285 | R54424 | Liver glutamate | 1.33 | 1.42 | 0.52 | 1.07 | 0.75 | 0.2 | 0.79 | 0.84 | 0.27 |
| | | dehydrogenase | | | | | | | | | |
| 45233 | H07880 | Chaperonin protein | 0.84 | 1.24 | 0.57 | 1.13 | 0.41 | 0.25 | 1 | 0.84 | 0.34 |
| | | (Tcp20) gene | | | | | | | | | |
| 814989 | AA465123 | Protein phosphatase 2C | 2.63 | 2.3 | 19.46 | 1.18 | 1.92 | 12.88 | 1.11 | 0.82 | 9.79 |
| | | gamma | | | | | | | | | |
| 825312 | AA504465 | ATP synthase, H+ | 1.22 | 1.13 | 7.79 | 1.56 | 1.52 | 9.77 | 1.08 | 0.82 | 6.37 |
| | | transporting, mitochondrial | | | | | | | | | |
| 138991 | R62603 | Homos sapien, alpha-3 | 1.18 | 0.87 | 0.45 | 2.42 | 1.21 | 0.58 | 0.99 | 0.82 | 0.31 |
| | | (VI) collagen | | | | | | | | | |
| 878798 | AA670408 | Beta-2microglobulin | 0.92 | 0.97 | 0.37 | 1.71 | 1 | 0.54 | 1.13 | 0.82 | 0.31 |
| | | precursor | | | | | | | | | |
| 878130 | AA775415 | SMT3B protein | 1.49 | 1.3 | 0.42 | 1.26 | 0.72 | 0.24 | 1.43 | 0.81 | 0.48 |
| 866874 | AA679345 | BTK region clone ftp-3 | 1.54 | 1.48 | 6.74 | 1.59 | 1.63 | 6.77 | 1.08 | 0.79 | 5.11 |
| 897641 | AA496792 | Heterogenous Nuclear | 0.66 | 0.57 | 0.35 | 0.44 | 0.64 | 0.32 | 0.71 | 0.79 | 0.58 |
| | | Ribonucleoprotein | | | | | | | | | |
| 24145 | R37953 | Adenylyl cyclase-associated | 0.77 | 0.57 | 0.27 | 0.66 | 0.45 | 0.22 | 1.05 | 0.78 | 0.41 |
| | | protein (CAP) | | | | | | | | | |
| 770027 | AA427433 | PP2A, 65 kDa alpha | 0.64 | 0.22 | 0.71 | 1.39 | 0.36 | 0.89 | 2.65 | 0.78 | 0.61 |
| 209841 | H67086 | TEB4 protein | 0.79 | 0.7 | 0.36 | 1.48 | 0.91 | 0.49 | 1.08 | 0.76 | 0.37 |
| 486221 | AA044059 | Voltage-dependent anion | 0.76 | 0.61 | 0.34 | 1.74 | 0.72 | 0.22 | 1.71 | 0.76 | 0.17 |
| | | channel 1 | | | | | | | | | |
| 840511 | AA486321 | Vimentin | 0.86 | 0.81 | 0.27 | 1.36 | 0.79 | 0.38 | 1.25 | 0.75 | 0.38 |
| 1461138 | AA868008 | H4/g gene H4 histone | 2.22 | 2.07 | 0.93 | 1.76 | 1.29 | 0.53 | 0.81 | 0.73 | 0.22 |
| 853151 | AA668301 | Ribosomal protein S16 | 1.13 | 0.98 | 0.18 | 1.67 | 0.85 | 0.42 | 1.27 | 0.73 | 0.37 |
| 810612 | AA464731 | Calgizzarin | 0.89 | 0.65 | 0.12 | 2.06 | 0.84 | 0.31 | 0.78 | 0.73 | 0.2 |
| 71116 | T50282 | Tissue factor pathway | 0.18 | 0.58 | 0.39 | 1.44 | 0.77 | 0.31 | 0.67 | 0.73 | 0.19 |
| | | inhibitor precursor | | | | | | | | | |
| 1471841 | AA873355 | ATPase, Na+/K+ transporting, | 0.5 | 0.52 | 0.14 | 1.18 | 1 | 0.36 | 0.81 | 0.72 | 0.36 |
| | | alpha 1 polypeptide | | | | | | | | | |
| 869450 | AA680244 | Ribosomal protein L11 | 0.77 | 0.64 | 0.29 | 1.29 | 0.75 | 0.24 | 1.36 | 0.72 | 0.37 |
| 119133 | T94169 | Stress-activated protein | 0.8 | 0.68 | 0.31 | 1.28 | 0.7 | 0.38 | 0.91 | 0.72 | 0.35 |
| | | kinase JNK1 | | | | | | | | | |
| 842784 | AA486200 | Phosphate carrier, | 0.87 | 0.92 | 0.42 | 1.55 | 1.08 | 0.68 | 0.87 | 0.71 | 0.38 |
| | | mitochondrial | | | | | | | | | |
| 869538 | AA680322 | NADH: ubiquinone oxido- | 0.41 | 0.75 | 0.13 | 1.36 | 0.62 | 0.6 | 1.77 | 0.71 | 0.24 |
| | | reductase MLRQ subunit | | | | | | | | | |
| 753862 | AA410517 | Cytoplasmic antiproteinase | 0.95 | 0.86 | 0.3 | 1.59 | 0.93 | 0.27 | 1.21 | 0.7 | 0.25 |
| 809992 | AA454852 | 55.11 binding protein | 0.68 | 0.93 | 0.5 | 1.21 | 1.02 | 0.49 | 1.23 | 0.69 | 0.3 |
| 32257 | R43360 | Signal recognition particle | 0.81 | 1 | 0.46 | 1.65 | 0.65 | 0.32 | 1.13 | 0.69 | 0.26 |
| | | 9 kDa protein | | | | | | | | | |
| 204257 | H59231 | Metalloprotease MDC9 | 0.84 | 0.72 | 0.13 | 0.61 | 0.5 | 0.32 | 1.43 | 0.69 | 0.24 |
| 745496 | AA625981 | FK506-binding protein 1 | 1.28 | 1.33 | 0.39 | 1 | 0.48 | 0.19 | 1.03 | 0.69 | 0.62 |
| | | (12 kDa) | | | | | | | | | |
| 149013 | R82300 | S-adenosylmethionine | 0.99 | 0.62 | 9.26 | 0.39 | 0.85 | 9.11 | 0.82 | 0.68 | 3.74 |
| | | decarboxylase 1 | | | | | | | | | |
| 266106 | N21624 | 14-3-3 epsilon | 0.78 | 0.84 | 0.32 | 0.99 | 0.54 | 0.38 | 1.03 | 0.68 | 0.2 |
| 813673 | AA453749 | Hepatoma-derived growth | 1.48 | 1.46 | 0.14 | 2.18 | 1.05 | 0.44 | 1.07 | 0.66 | 0.15 |
| | | factor | | | | | | | | | |
| 714106 | AA284669 | Urokinase-type plasminogen | 0.55 | 0.29 | 0.15 | 1.58 | 0.85 | 0.47 | 0.66 | 0.66 | 0.31 |
| | | activator | | | | | | | | | |
| 486110 | AA040703 | Profilin 2 | 0.6 | 0.7 | 0.24 | 1.31 | 0.78 | 0.31 | 1.09 | 0.66 | 0.3 |
| 837904 | AA434088 | Ribosomal protein L 10 | 0.71 | 0.46 | 0.06 | 0.88 | 0.55 | 0.1 | 0.79 | 0.66 | 0.24 |
| 813983 | AA455640 | Signalosome subunit 3 (Sgn3) | 0.89 | 06 | 5.41 | 0.71 | 0.44 | 8.71 | 1.39 | 0.66 | 6.55 |
| 897596 | AA496880 | Ribsomal protein L5 | 0.68 | 0.8 | 0.26 | 1.69 | 0.86 | 0.48 | 1.19 | 0.65 | 0.21 |
| 40017 | R52654 | Cytochrome c-1 | 0.62 | 0.77 | 0.24 | 0.93 | 0.57 | 0.26 | 0.85 | 0.65 | 0.32 |
| 878681 | AA775364 | 60S Ribosomal protein L30 | 0.88 | 0.74 | 0.34 | 1.29 | 0.73 | 0.38 | 1.22 | 0.64 | 0.25 |
| 511459 | AA115309 | Probable Protein Disulfide | 0.88 | 1.06 | 0.35 | 1.13 | 0.79 | 0.32 | 0.93 | 0.63 | 0.24 |
| | | Isomerase E1 | | | | | | | | | |
| 85171 | T71316 | ADP-ribosylation factor 4 | 0.76 | 0.82 | 0.39 | 0.94 | 0.66 | 0.25 | 0.84 | 0.62 | 0.19 |
| 741067 | AA478436 | SWI/SNF complex 60 kDa | 0.62 | 0.7 | 0.19 | 0.81 | 0.58 | 0.23 | 0.77 | 0.62 | 0.36 |
| | | subunit (BAF60b) | | | | | | | | | |
| 884546 | AA629808 | Ribosomal protein L6 | 0.61 | 0.71 | 0.01 | 1.32 | 0.75 | 0.04 | 1.15 | 0.61 | 0.09 |
| 650578 | AA608515 | NADH ubiquinone oxidore- | 0.55 | 0.77 | 0.34 | 0.9 | 0.37 | 0.4 | 0.39 | 0.6 | 0.14 |
| | | ductase subunit B13 (B13) | | | | | | | | | |
| 433666 | AA699317 | Testican | 0.52 | 0.68 | 0.49 | 0.71 | 0.31 | 0.33 | 1.15 | 0.6 | 0.75 |
| 43550 | H05914 | Lactate dehydrogenase-A | 0.845 | 0.679 | 0.255 | 1.175 | 0.755 | 0.35 | 0.995 | 0.59 | 0.25 |
| | | (LDH-A, EC 11.27) | | | | | | | | | |
| 79688 | T62529 | snRNP core protein Sm D2 | 0.73 | 0.3 | 0 | 2.37 | 0.61 | 0 | 2.94 | 0.59 | 0 |
| 897806 | AA598526 | MOP1 | 0.6 | 0.45 | 0.38 | 0.77 | 0.54 | 0.33 | 0.6 | 0.59 | 0.19 |
| 703581 | AA278759 | Hematopoietic proteoglycan | 0.2 | 0.37 | 0.42 | 0.94 | 0.29 | 0.23 | 0.76 | 0.59 | 0.24 |
| | | core protein | | | | | | | | | |
| 34355 | R44288 | Calmodulin | 0.65 | 0.705 | 0.2 | 1.315 | 0.565 | 0.17 | 1.1 | 0.585 | 0.25 |
| 484333 | AA703141 | Protein 4.1 | 0.49 | 0.54 | 0.22 | 0.71 | 0.52 | 0.27 | 0.75 | 0.58 | 0.3 |
| 884718 | AA629567 | Heat shock cognate 71 kDa | 0.7 | 0.64 | 0.17 | 1.66 | 0.66 | 0.2 | 0.88 | 0.56 | 0.2 |
| | | protein | | | | | | | | | |
| 511586 | AA126911 | Heterogeneous nuclear | 1.08 | 0.93 | 0.25 | 1.19 | 0.66 | 0.35 | 1.02 | 0.55 | 0.31 |
| | | ribonucleoprotein A1 | | | | | | | | | |
| 131382 | R22977 | Moesin | 1.14 | 1.8 | 0.5 | 2.11 | 1.06 | 0.4 | 0.83 | 0.54 | 0.22 |
| 66686 | T67270 | UBI-CYT C reductase VI | 0.8 | 0.71 | 0.29 | 1.1 | 0.61 | 0.3 | 0.81 | 0.53 | 0.28 |
| 26099 | R37286 | hnRNP core protein A1 | 0.72 | 0.68 | 0.25 | 1.24 | 0.65 | 0.05 | 0.88 | 0.51 | 0.1 |
| 856961 | AA669674 | Int-6 | 1.58 | 1.13 | 0.1 | 1.01 | 0.57 | 0.5 | 0.72 | 0.49 | 0.32 |
| 857243 | AA629641 | Ribosomal protein S13 | 0.56 | 0.8 | 0 | 1.14 | 0.33 | 0 | 1.16 | 0.49 | 0 |
| 950574 | AA608514 | Histone H3.3 | 2.66 | 2.74 | 1.11 | 1.8 | 1.75 | 0.56 | 0.91 | 0.48 | 0.22 |
| 725877 | AA292410 | Clusterin | 0.83 | 0.58 | 4.08 | 1.23 | 0.91 | 4.33 | 0.45 | 0.48 | 3.41 |
| 843098 | AA488676 | Neuronal tissue-enriched | 1.51 | 0.96 | 0.86 | 1.66 | 0.64 | 0.16 | 0.86 | 0.48 | 0.29 |
| | | acidic protein (NAP-22) | | | | | | | | | |
| 753862 | AA411343 | Ribosomal protein S29 | 0.75 | 0.68 | 0.34 | 1.24 | 0.56 | 0.37 | 1.03 | 0.48 | 0.27 |
| 33525 | R43973 | Elongation factor-1-gamma | 0.735 | 0.515 | 0.165 | 1.325 | 0.75 | 0.335 | 0.795 | 0.47 | 0.215 |
| 853938 | AA644679 | Cytoplasmic dynein light | 0.23 | 0.48 | 0.15 | 1.3 | 0.67 | 0.16 | 0.69 | 0.47 | 0.25 |
| | | chain 1 (hdlc 1) | | | | | | | | | |
| 1474174 | AA936799 | Matrix metalloproteinase 2 | 0.59 | 0.76 | 0.24 | 3.91 | 2.05 | 0.75 | 0.74 | 0.44 | 0.26 |
| 950489 | AA599127 | Superoxide dismutase 1 | 0.81 | 0.63 | 0.18 | 2.31 | 0.97 | 0.42 | 0.68 | 0.44 | 0.33 |
| | | (Cu/Zn) | | | | | | | | | |
| 490947 | AA136533 | Transcription elongation | 0.62 | 0.72 | 0.24 | 1.99 | 0.93 | 0.14 | 1.49 | 0.44 | 0.35 |
| | | factor B (SIII) | | | | | | | | | |
| 29054 | R40850 | Alpha-centractin | 0.685 | 0.495 | 0.13 | 2.12 | 1.2 | 0.435 | 0.655 | 0.43 | 0.24 |
| 843134 | AA485909 | Prostatic binding protein | 3.26 | 2.25 | 0 | 1.78 | 1.18 | 0 | 1.34 | 0.42 | 0 |
| 1473289 | AA916327 | Protective protein beta- | 0.82 | 0.46 | 0.18 | 1.63 | 0.95 | 0.42 | 0.43 | 0.37 | 0.27 |
| | | galactosidase | | | | | | | | | |
| 782449 | AA431440 | hnRNP-E2 | 0.88 | 0.62 | 0.17 | 1.51 | 0.76 | 0.22 | 1.26 | 0.37 | 0.07 |
| 34255 | R44202 | Catechol-O-methyltransferase | 1.8 | 1 | 0.58 | 1.55 | 0.765 | 0.115 | 1.35 | 0.36 | 0.195 |
| | | (COMT) | | | | | | | | | |

1. A method of selecting an approach to treating cancer in a subject using radiation therapy, said method comprising (i) analyzing the level of expression of a cancer-associated gene in a sample comprising cancer cells from the subject, and (ii) selecting a type, schedule, route, and/or amount of radiation therapy for treating the subject based on the results of the analysis.

2. The method of claim 1, wherein the subject has previously been treated using radiation therapy.

3. The method of claim 1, wherein the subject has not been previously treated using radiation therapy.

4. The method of claim 1, wherein the subject has previously received cancer treatment not involving radiation therapy.

5. The method of claim 1, wherein an increase in expression of a gene associated with resistance to radiation therapy, or a decrease in expression of a gene associated with sensitivity to radiation therapy, is detected and, based on detection of said increase or said decrease, administration of a radiosensitizer is selected for treatment of the subject.

6. The method of claim 5, wherein the time frame during which said radiosensitizer is to be administered to the subject is determined by analysis of the temporal expression of said gene associated with resistance to radiation therapy or said gene associated with sensitivity to radiation therapy.

7. The method of claim 5, wherein the dosage at which said radiosensitizer is to be administered to the subject is determined by analysis of the level of expression of said gene associated with resistance to radiation therapy or said gene associated with sensitivity to radiation therapy.

8. The method of claim 1, wherein an increase in expression of a gene associated with sensitivity to radiation therapy, or a decrease in expression of a gene associated with resistance to radiation therapy, is detected, indicating treatment using further radiation therapy.

9. A method of selecting an approach to treating cancer in a subject that has previously been treated using radiation therapy, said method comprising (i) analyzing the level of expression of a cancer-associated gene in a sample comprising cancer cells from said subject, and (ii) selecting a type, schedule, route, and/or amount of a therapy not involving further radiation therapy for treating the subject based on the results of the analysis.

10. The method of claim 4 or 9, wherein the non-radiation therapy is selected from the group consisting of chemotherapy, biological therapy, gene therapy, oncolytic viral therapy, and surgery.

11. The method of claim 1 or 9, wherein the expression of more than one cancer-associated gene is analyzed.

12. The method of claim 11, wherein the analysis is carried out using a nucleic acid molecule array.

13. The method of claim 2, 4, or 9, wherein the analysis comprises determination of the level of expression of a gene at more than one time point after the prior treatment, and analysis of the time course of changes in the level of expression of the gene indicates an optimal time frame during which a particular type of subsequent treatment should be carried out.

14. The method of claim 2, 4, or 9, wherein the analysis comprises analyzing the effects of varying doses of the prior treatment, and analysis of the effect of the varying doses on the level of expression of the gene indicates an optimal dosage at which a particular type of subsequent treatment should be carried out.

15. The method of claim 2, 4, or 9, wherein the prior treatment is carried out on a tumor sample ex vivo.

16. The method of claim 2, 4, or 9, wherein the prior treatment is carried out in vivo.

17. A method of treating cancer in a subject, said method comprising using an approach selected by using the method of claim 1 or 9.

18. A kit for use in selecting an approach to treating cancer in a subject, said kit comprising (i) a cancer-associated gene probe, and (ii) instructions to hybridize the probe with nucleic acid molecules derived from a subject tumor sample, to determine the level of expression of the gene in the tumor as an indication of an appropriate type, schedule, and/or amount of therapy to use in the treatment.

19. The kit of claim 18, wherein the kit comprises more than one cancer-associated gene probe.

20. The kit of claim 18, wherein the cancer-associated gene probe is immobilized on a solid support.

21. The kit of claim 20, wherein the solid support comprises an array of probes.

22. The kit of claim 18, further comprising reagents and buffers that can be used to carry out said hybridization and/or in the determination of the level of expression of said gene.

23. A method of identifying a gene that can be used in the identification of an approach to treating cancer in a subject, said method comprising contacting a nucleic acid molecule array with cDNA or RNA derived from a sample from a tumor of said subject and detecting altered levels of binding of the tumor sample-derived cDNA or RNA to a position in the array, relative to a control, and determining the identity of the gene that corresponds to the position in the array.
